(19) United States
(12) Patent Application Publication
(10) Pub. No.: US 2004/0254351 A1
(43) Pub. Date: Dec. 16, 2004

(54) Hyperglycosylated polypeptides

(76) Inventors: John Michael Beals, Indianapolis, IN (US); Joe Christopher Berry, Greenwood, IN (US); Marcia Kay Jones, Franklin, IN (US); Uma Kuchibhotla, Indianapolis, IN (US); Radhakrishnan Rathnachalam, Carmel, IN (US)

Correspondence Address: ELI LILLY AND COMPANY, PATENT DIVISION, P.O. BOX 6288, INDIANAPOLIS, IN, 46206-6288, US

(21) Appl. No.: 10/362,385

(22) PCT Filed: Aug. 24, 2001
(86) PCT No.: PCT/US01/22622

Related U.S. Application Data
(60) Provisional application No. 60/231,174, filed on Sep. 7, 2000.

(51) Int. Cl. C07K 14/535

(52) U.S. Cl. 530/351

(57) ABSTRACT

The present invention addresses the need for better pharmaceutical agents for treating patients that have reduced circulating levels of neutrophilic granulocytes, such as after chemotherpay regimens or in chronic congenital neutropneia by providing novel biological active glycosylated G-CSF analogs.

The present invention is in the field of human medicine, particularly in the treatment of conditions treatable by stimulation of circulating neutrophils, such as after chemotherapy regimens or in chronic congenital neutropenia. More specifically, the invention relates to novel glycosylated proteins with granulocyte-colony stimulating factor activity.

Among all blood cell lineages, the modulation of neutrophil and platelet production has been of highest interest to clinical oncologists and hematologists. Myelosuppression is the single most severe complication of cancer chemotherapy, and a major cause of treatment delay during multiple-cycle or combination chemotherapy. It is also the major dose-limiting factor for most chemotherapeutic agents. Due to the short half-lives of neutrophils in peripheral blood, life-threatening falls in neutrophil levels are seen after a number of conventional anti-tumor chemotherapy regimens.

The most prominent regulator of granulopoiesis is granulocyte-colony stimulating factor (G-CSF). G-CSF induces proliferation and differentiation of hematopoietic progenitor cells resulting in increased numbers of circulating neutrophils. G-CSF also stimulates the release of mature neutrophils from bone marrow and activates their functional state. [Souza L. M., et al. (1986) Science 232:61-65]. Thus, therapeutic proteins with G-CSF activity have tremendous value in situations where there are reduced circulating levels of neutrophilic granuloctyes.

Like other cytokines and growth factors, G-CSF exerts its effects by binding to a specific receptor displayed on the surface of target cells. The interaction of G-CSF with its receptor induces intracellular events that stimulate proliferation and terminal differentiation of neutrophilic granulocytes from progenitor cells that present the G-CSF receptor. G-CSF controls the proliferation of the committed progenitor cells (CFU-GM), their maturation into CFU-G, and ultimately their maturation into functional mature neutrophils.

The natural human G-CSF protein exists in two forms of 174 and 177 amino acids. [Nagata S., et al. (1986) EMBO J. 5:575-581]. The more abundant and more active 174 amino acid form has been used in the development of pharmaceutical products by recombinant DNA technology. The crystal structure of G-CSF indicates that it is a member of the four-helix bundle structural superfamily of growth factors: helix A (residues 11-39), helix B (residues 71-91), helix C (residues 100-123), and helix D (residues 143-172). [Hill, C. P., et al. (1993) Proc. Nat. Acad. Sci. 90:5167-5171]. Native G-CSF has an O-linked carbohydrate moiety attached to residue 133 when expressed in certain-mammalian cells. However, O-linked glycosylation does not appear critical for activity in vitro or in vivo.

Several G-CSF therapeutic products are currently available. Lenograstim® is a recombinant form of human G-CSF produced in Chinese Hamster Ovary (CHO) cells. It is indistinguishable from the 174 amino acid natural form of human G-CSF. Filgrastim® is a recombinant form of human G-CSF produced in an E. coli expression system and, therefore, is not glycosylated. In addition, there is a G-CSF analog known as Nartograstim® which is also produced in E. coli. This analog has 5 mutations in addition to a N-terminal methionine. The E. coli derived wild-type molecule has limited stability in solution compared to the CHO derived molecule. It is thought that the O-linked sugar chain of G-CSF produced in mammalian cells protects the protein from polymerization and denaturation. However, there does not appear to be any clinical or therapeutic consequences of producing the compound in CHO versus E. coli. [Oh-Eda, M., et al. (1990) J. Biol. Chem. 265:11432-11435].

All three compounds have a short plasma half-life. Thus, they must be administered intravenously or subcutaneously at fairly frequent intervals (once or twice a day) in order to maintain their neutrophil stimulating properties. In addition, this short half-life limits the performance of the drug to traditional drug delivery systems. It would clearly benefit the treatment of patients with abnormally low neutrophils, and reduce the discomfort and inconvenience associated with frequent injections to provide a pharmaceutical agent that could be administered less frequently and optionally by alternative routes of administration. Thus, a need exists to develop agents that stimulate the production of mature neutrophils and are more optimal in their duration of effect.

One approach is to alter the carbohydrate content of G-CSF by substituting amino acids that can act as substrates for glycosylating enzymes in mammalian cells. There are two types of glycosylation that occur in mammalian cells. N-linked glycosylation occurs when carbohydrate chains are bound to Asn residues in some proteins and O-linked glycosylation occurs when carbohydrate chains are bound to Ser or Thr residues in some proteins.

N-glycosylated carbohydrate chains have a common basic core structure composed of five monosaccharide residues, namely two N-acetylglucosamine residues and three mannose residues. The carbohydrate chain is transferred to an Asn residue in an amino acid sequence such as Asn-X-Ser/Thr wherein X is any amino acid except Pro and whereby an N-acetylglucosamine linkage is formed. This reaction is catalyzed by an oligosaccharyl transferase. Many proteins contain an Asn-X-Ser/Thr sequence; however, not all of these proteins are glycosylated. Thus, the presence of this sequence alone is generally not enough to ensure that glycosylation occurs. The three dimensional structure as well as the location of the consensus site in the sequence can be critical in inducing binding of a carbohydrate chain.

The role of carbohydrates is complex. Studies show that for proteins that are normally glycosylated in vivo, such as erythropoietin, proper glycosylation is critical for proper biosynthesis and secretion of the protein. It also may promote correct folding during protein expression and possibly protect the protein from degradation during biosynthesis, secretion, and circulation. However, for proteins that are not normally glycosylated or are not heavily glycosylated, such as G-CSF, it is difficult to predict what effect glycosylation will have on activity of the protein. Furthermore, it is impossible to predict for these types of proteins where, specifically in the protein, changes can be made to induce glycosylation and whether this glycosylation will negatively affect receptor binding, stability, activity, or half-life.

There is no published data showing that adding specific glycosylation sites to G-CSF extends its half-life in vivo. Katsutoshi et al. describe a particular glycosylated G-CSF analog that may be more resistant to protease activity [U.S. Pat. No. 5,218,092]. This analog has the same backbone as Nartograstim® (N-terminal Met (−1), Thr1Ala, Leu3Thr, Gly4Tyr, Pro5Arg, Cys17Ser) with a glycosyaltion site introduced at position 144 (Phe145Asn, Arg147Ser).

While Katsutoshi, et al. describe generally different roles that a carbohydrate chain may play when linked to a protein like G-CSF, they do not provide activity data for any glycosylated G-CSF analog nor do they characterize any analogs with the exception of the single analog described above. In fact, Katsutohi et al. state that regardless of whether the three-dimensional structure is known or unknown, it is necessary to actually introduce carbohydrate addition sites into specific areas of the protein to determine whether glycosylation can actually take place at that site and if so whether the glycosylated protein retains activity.

The focus of the Katsutoshi, et al. application was to create an analog that would be less likely to be degraded by endogenous proteases due to an introduced glycosylation site near a sequence commonly cleaved by proteases. However, protecting a protein from degradation in this manner is merely one way to try and extend the half-life of the protein. Glycosylation may also be introduced to effect clearance mechanisms for a particular protein. G-CSF is cleared mainly by receptor mediated endocytosis, but there appears to be significant renal clearance as well. Renal clearance can be decreased by increasing the size of a protein and/or increasing the negative charge on the protein at physiological pH. Both of these can be accomplished by introducing glycosylation sites that have sialic acid moietes attached to them. Thus, the present invention provides G-CSF analogs wherein glycosylation sites have been introduced at specific regions of G-CSF in order to enhance the biological activity of G-CSF in vivo. The present invention provides data showing that these analogs are glycosylated in mammalian cells and retain their activity.

One aspect of the present invention includes glycosylated proteins of the Formula (I) [SEQ ID NO:1]

| | | | | | | | | | | | | | | | | |
|---|---|---|---|---|---|---|---|---|---|---|---|---|---|---|---|---|
| 1 | | | | 5 | | | | | 10 | | | | | 15 | | |
| Thr | Pro | Leu | Gly | Pro | Ala | Ser | Ser | Leu | Pro | Gln | Ser | Phe | Leu | Leu | Lys | (I) |
| | | | 20 | | | | | 25 | | | | | 30 | | | |
| Xaa | Leu | Glu | Gln | Val | Arg | Lys | Ile | Gln | Gly | Asp | Gly | Ala | Ala | Leu | Gln | |
| | | 35 | | | | | 40 | | | | | 45 | | | | |
| Glu | Lys | Leu | Cys | Xaa | Xaa | Xaa | Lys | Leu | Cys | His | Pro | Glu | Glu | Leu | Val | |
| | 50 | | | | | 55 | | | | | 60 | | | | | |
| Leu | Leu | Gly | His | Ser | Leu | Gly | Ile | Xaa | Xaa | Xaa | Xaa | Xaa | Xaa | Xaa | Xaa | |
| 65 | | | | | 70 | | | | | 75 | | | | | 80 | |
| Xaa | Xaa | Xaa | Xaa | Xaa | Gln | Leu | Ala | Gly | Cys | Leu | Ser | Gln | Leu | His | Ser | |
| | | | | 85 | | | | | 90 | | | | | 95 | | |
| Gly | Leu | Phe | Leu | Tyr | Gln | Gly | Leu | Leu | Gln | Ala | Leu | Xaa | Xaa | Xaa | Ser | |
| | | | 100 | | | | | 105 | | | | | 110 | | | |
| Xaa | Glu | Leu | Gly | Pro | Thr | Leu | Asp | Thr | Leu | Gln | Leu | Asp | Val | Ala | Asp | |
| | | 115 | | | | | 120 | | | | | 125 | | | | |
| Phe | Ala | Thr | Thr | Ile | Trp | Gln | Gln | Met | Glu | Glu | Leu | Gly | Met | Ala | Pro | |
| | 130 | | | | | 135 | | | | | 140 | | | | | |
| Ala | Leu | Gln | Pro | Xaa | Xaa | Xaa | Ala | Met | Pro | Ala | Phe | Xaa | Xaa | Xaa | Phe | |
| 145 | | | | | 150 | | | | | 155 | | | | | 160 | |
| Gln | Arg | Arg | Ala | Gly | Gly | Val | Leu | Val | Ala | Ser | His | Leu | Gln | Ser | Phe | |
| | | | | 165 | | | | | 170 | | | | | | | |
| Leu | Glu | Val | Ser | Tyr | Arg | Val | Leu | Arg | His | Leu | Ala | Gln | Pro | | | |

wherein:

Xaa at position 17 is Cys, Ala, Leu, Ser, or Glu;

Xaa at position 37 is Ala or Asn;

Xaa at position 38 is Thr, or any other amino acid except Pro;

Xaa at position 39 is Tyr, Thr, or Ser;

Xaa at position 57 is Pro or Val;

Xaa at position 58 is Trp or Asn;

Xaa at position 59 is Ala or any other amino acid except Pro;

Xaa at position 60 is Pro, Thr, Asn, or Ser,

Xaa at position 61 is Leu, or any other amino acid except Pro;

Xaa at position 62 is Ser or Thr;

Xaa at position 63 is Ser or Asn;

Xaa at position 64 is Cys or any other amino acid except Pro;

Xaa at position 65 is Pro, Ser, or Thr;

Xaa at position 66 is Ser or Thr;

Xaa at position 67 is Gln or Asn;

Xaa at position 68 is Ala or any other amino acid except Pro;

Xaa at position 69 is Leu, Thr, or Ser

Xaa at position 93 is Glu or Asn

Xaa at position 94 is Gly or any other amino acid except Pro;

Xaa at position 95 is Ile, Asn, Ser, or Thr;

Xaa at position 97 is Pro, Ser, Thr, or Asn;

Xaa at position 133 is Thr or Asn;

Xaa at position 134 is Gln or any other amino acid except Pro;

Xaa at position 135 is Gly, Ser, or Thr

Xaa at position 141 is Ala or Asn;

Xaa at position 142 is Ser or any other amino acid

except Pro; and

Xaa at position 143 is Ala, Ser, or Thr;

and wherein:

Xaa at positions 37, 38, and 39 constitute region 1;

Xaa at positions 58, 59, and 60 constitute region 2;

Xaa at positions 59, 60, and 61 constitute region 3;

Xaa at positions 60, 61, and 62 constitute region 4;

Xaa at positions 61, 62, and 63 constitute region 5;

Xaa at positions 62, 63, and 64 constitute region 6;

Xaa at positions 63, 64, and 65 constitute region 7;

Xaa at positions 64, 65, and 66 constitute region 8;

Xaa at positions 67, 68, and 69 constitute region 9;

Xaa at positions 93, 94, and 95 constitute region 10;

Xaa at positions 94, 95, and Ser at position 96 constitute region 11;

Xaa at positions 95, and 97, and Ser at position 96 constitute region 12;

Xaa at positions 133, 134, and 135 constitute region 13;

Xaa at positions 141, 142, and 143 constitute region 14;

and provided that at least one of regions 1 through 14 comprises the sequence Asn Xaa1 Xaa2 wherein Xaa1 is any amino acid except Pro and Xaa2 is Ser or Thr.

Thus, the glycosylated proteins of the present invention include analogs wherein one or any combination of two or more regions comprise the sequence Asn Xaa1 Xaa2 wherein Xaa1 is any amino acid except Pro and Xaa2 is Ser or Thr.

Preferred glycosylated proteins include the following:

a) G-CSF[A37N,Y39T]

b) G-CSF[P57V,W58N,P60T]

c) G-CSF[P60N,S62T]

d) G-CSF[S63N,P65T]

e) G-CSF[Q67N,L69T]

f) G-CSF[E93N,I95T]

g) G-CSF[T133N,G135T]

h) G-CSF[A141N,A143T]

i) G-CSF[A37N,Y39T,P57V,W58N,P60T]

j) G-CSF[A37N,Y39T,P60N,S62T]

k) G-CSF[A37N,Y39T,S63N,P65T]

l) G-CSF[A37N,Y39T,Q67N,L69T]

m) G-CSF[A37N,Y39T,E93N,I95T]

n) G-CSF[A37N,Y39T,T133N,G135T]

o) G-CSF[A37N,Y39T,A141N,A143T]

p) G-CSF[A37N,Y39T,P57V,W58N,P60T,S63N,P65T]

q) G-CSF[A37N,Y39T,P57V,W58N,P60T,Q67N,L69T]

r) G-CSF[A37N,Y39T,S63N,P65T,E93N,I95T]

The present invention also includes glycosylated proteins which are the product of the expression in a host cell of an exogenous DNA sequence which comprises a DNA sequence encoding a protein of Formula I described above.

The present invention includes an isolated nucleic acid sequence, comprising a polynucleotide encoding a glycosylated protein described above. Exemplary isolated nucleic acids of the present invention include isolated nucleic acid sequence comprising a polynucleotide selected from the group consisting of:

a) SEQ ID NO:2
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC AAC CAG ACC GCC ATG CCG GCC TTC GCC TCT GCT TTC
CGG GAC GTC GGG TTG GTC TGG CGG TAC GGC CGG AAG CGG AGA CGA AAG
CZG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
b) SEQ ID NO:3
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
c) SEQ ID NO:4
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
d) SEQ ID NO:5
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
e) SEQ ID NO:6
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
f) SEQ ID NO:7
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
g) SEQ ID NO:8
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
h) SEQ ID NO:9
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
i) SEQ ID NO:10
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
j) SEQ ID NO:11
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
k) SEQ ID NO:12
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
l) SEQ ID NO:13
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
m) SEQ ID NO:14
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
n) SEQ ID NO:15
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
o) SEQ ID NO:16
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG

The present invention includes vectors comprising a polynucleotide encoding a protein of Formula I described above as well as host cells comprising these vectors. The present invention also includes a process for producing a glycosylated protein comprising the steps of transcribing and translating a polynucleotide described above under conditions wherein the protein is glycosylated and expressed in detectable amounts.

The present invention encompasses a method for increasing neutrophil levels in a mammal comprising the administration of a therapeutically effective amount of a glycosylated protein described above. The present invention also includes the use of the glycosylated proteins described above for the manufacture of a medicament for the treatment of patients with insufficient circulating neutrophil levels.

The present invention also encompasses a pharmaceutical formulation adapted for the treatment of patients with insufficient neutrophil levels comprising a glycosylated protein as described above.

The invention is further illustrated with reference to the following drawings:

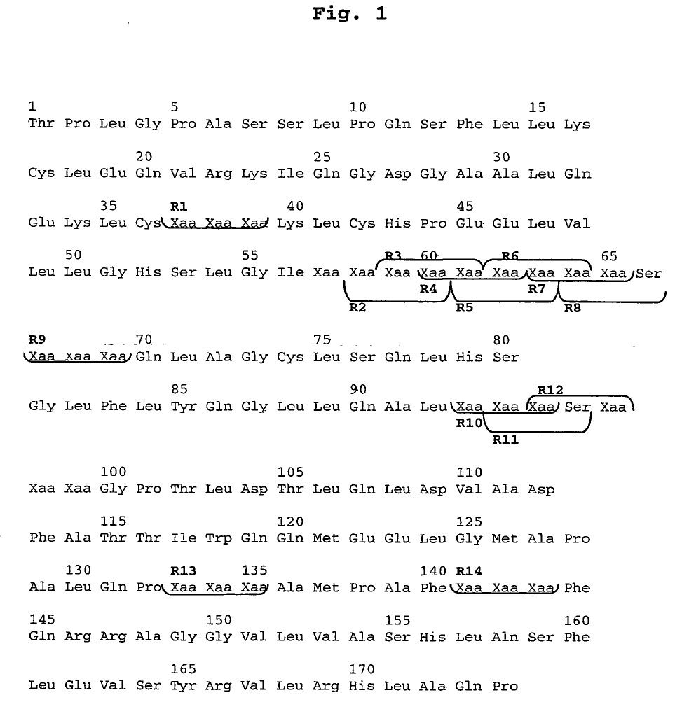

FIG. 1: Schematic illustrating nine regions in human G-CSF wherein the amino acid sequence can be mutated to create functional glycosylation sites.

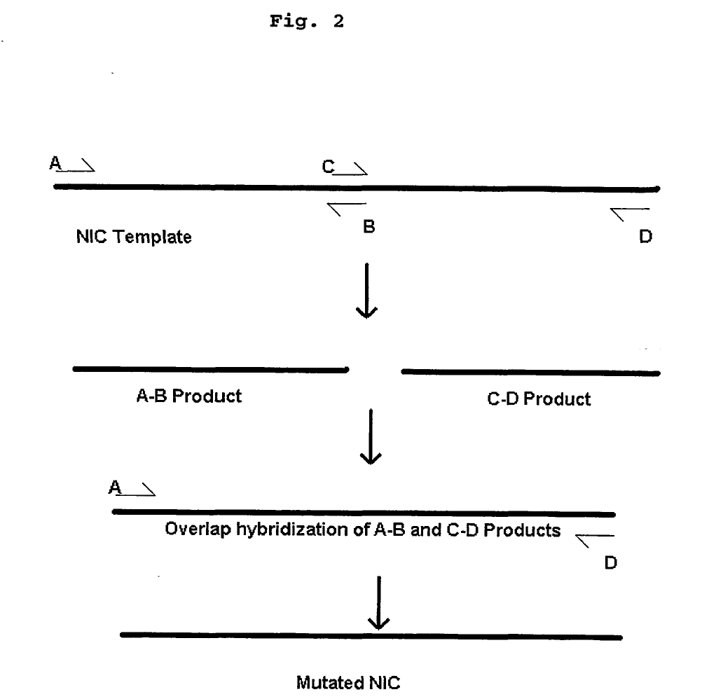

FIG. 2: Schematic illustrating the process of DNA mutagenesis by strand overlapping PCR.

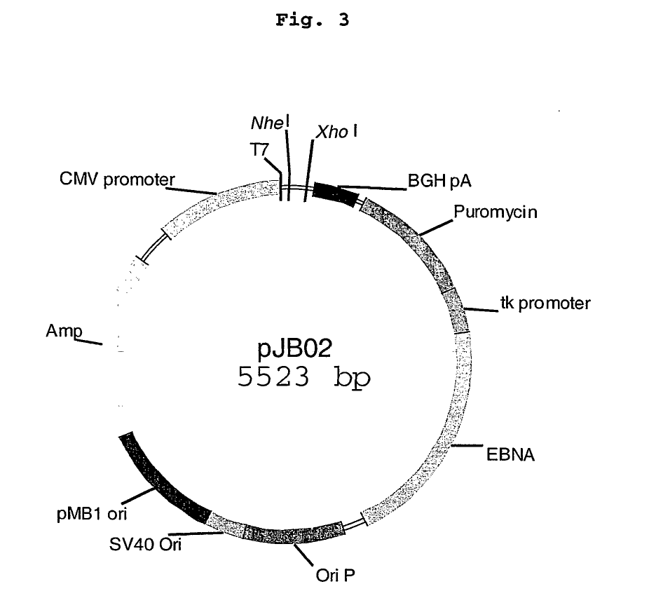

FIG. 3: Schematic of the 293/EBNA expression vector pJB02.

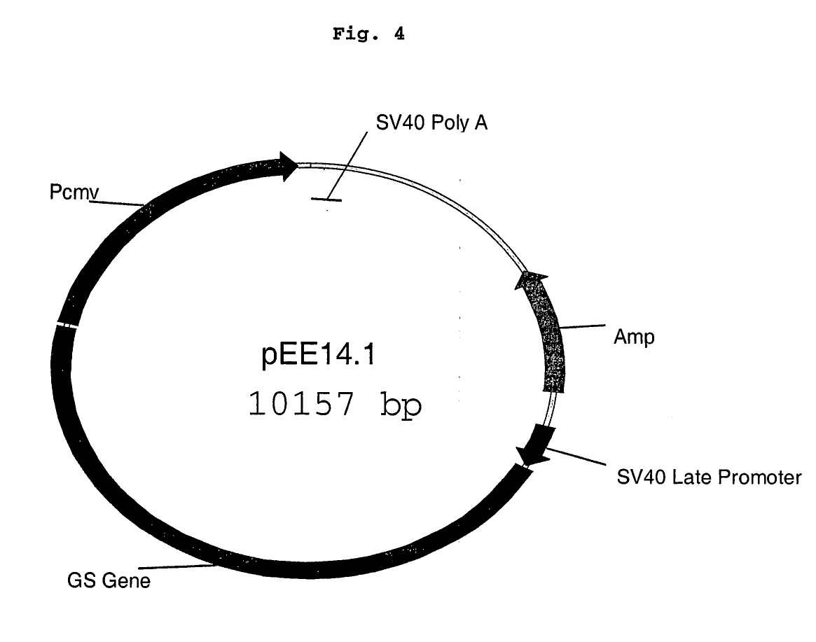

FIG. 4: Schematic of the CHO-K1 expression vector pEE14.1.

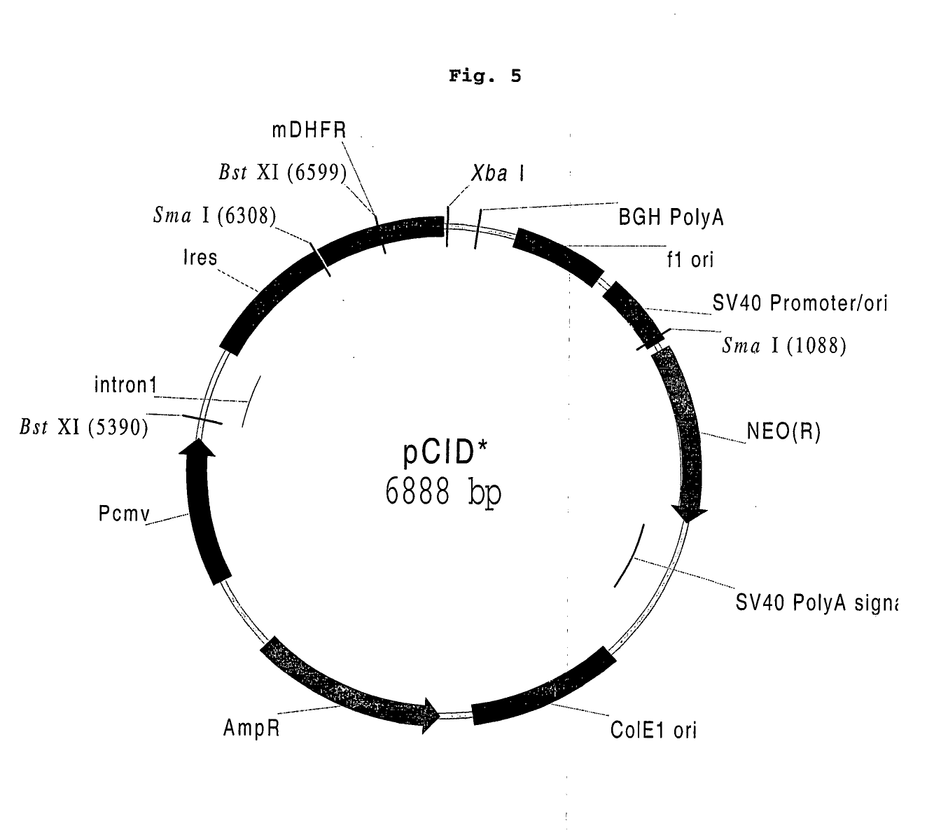

FIG. 5: Schematic of the CHO-DG44 expression vector PCID.

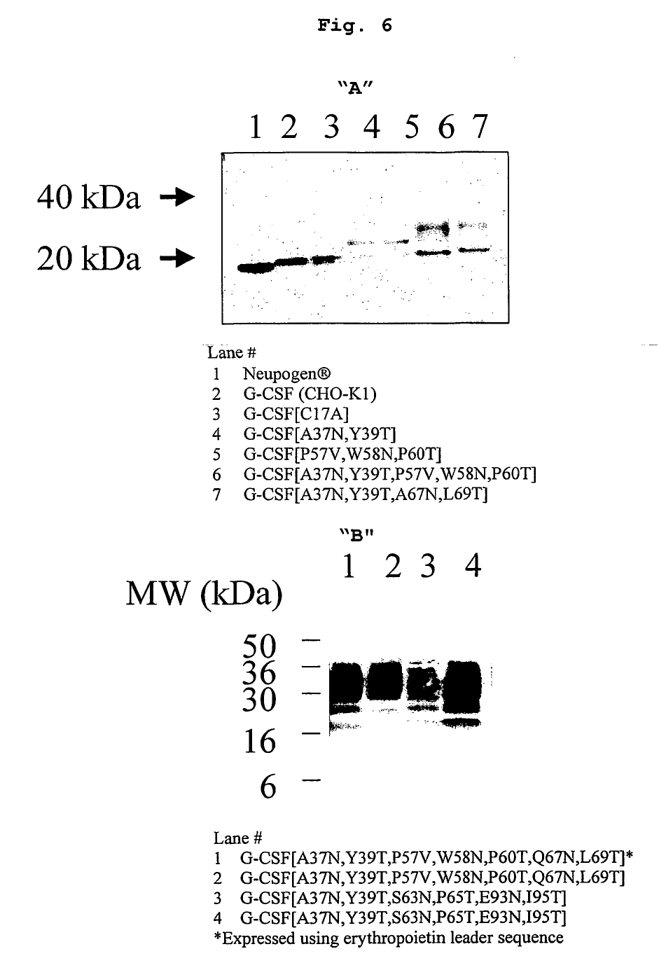

FIG. 6: SDS-PAGE analysis of glycosylated G-CSF analogs.

Encompassed by the invention are certain glycosylated analogs of G-CSF. Analogs of G-CSF refer to human G-CSF with one or more changes in the amino acid sequence which result in an increase in the number of sites for carbohydrate attachment compared with native human G-CSF expressed in animal cells in vivo. In addition, G-CSF analogs include human G-CSF wherein the O-linked glycosylation site at position 133 is replaced with an N-linked glycosylation site. Analogs are generated by site directed mutagenesis having substitution of amino acid residues creating new sites that are available for glycosylation. Analogs having a greater carbohydrate content than that found in native human G-CSF are generated by adding glycosylation sites that do not perturb the secondary, tertiary, and quaternary structure required for activity. Furthermore, because the glycosylated analogs of the present invention have a larger mass and an increased negative charge compared to native G-CSF, they will not be as rapidly cleared from the circulation.

It is preferred that the G-CSF analog have 1, 2, 3, or 4 additional sites for N-glycosylation. FIG. 1 illustrates fourteen different regions that can be glycosylated with very little effect on in vitro activity. Each region may be mutated to the consensus site for N-glycosylation addition which is Asn X1 X2 wherein X1 is any amino acid except Pro and X2 is Ser or Thr. It is preferred that the X1 amino acid be any other amino acid except Trp, Asp, Glu, or Leu and it is most preferred that the X1 amino acid be the naturally occurring amino acid. The scope of the present invention includes analogs wherein a single region (1 through 14) is mutated or wherein a region is mutated in combination with one or more other regions.

Analogs having carbohydrate attached to only a single mutated site have been expressed, purified, characterized, and tested for activity. Similarly analogs with multiple glycosylation sites have been expressed, purified, characterized, and tested for activity. For example G-CSF[A37N,Y39T] is G-CSF wherein the amino acids at positions 37 and 39 have been substituted to create a glycosylation site. This site of carbohydrate attachment is illustrated as region one in FIG. 1. G-CSF[A37N,Y39T,P57V,W58N,P60T] is an example of a G-CSF compound wherein amino acids in region 1 and region 2 are mutated to provide two functional glycosylation sites on a single molecule (FIG. 1). G-CSF[A37N,Y39T,P57V,W58N,P60T,Q67N,L69T] is an example of a G-CSF analog wherein the amino acids in region 1, region 2, and region 9 are mutated to provide three functional glycosylation sites on a single molecule (FIG. 1).

The present invention also encompasses G-CSF analogs wherein the O-linked glycosylation site at position 133 is mutated to serve as an N-linked glycosylation site. The N-linked carbohydrate will generally have a higher sialic acid content which will stabilize the protein and protect it from the rapid clearance mechanisms associated with native G-CSF.

The functions of a carbohydrate chain greatly depends on the structure of the attached carbohydrate moiety. Typically compounds with a higher sialic acid content will have better stability and longer half-lives in vivo. The N-linked oligosaccharides contain sialic acid in both an α2,3 and an α2,6 linkage to galactose. [Takeuchi et al. (1988) J. Biol. Chem. 263:3657]. Typically the sialic acid in the α2,3 linkage is added to galactose on the mannose α1,6 branch and the sialic acid in the α2,6 linkage is added to the galactose on the mannose α1,3 branch. The enzymes that add these sialic acids (β-galactoside α2,3 sialyltransferase and β-galactoside α2,6 sialyltransferase) are most efficeint at adding sialic acid to the mannose α1,6 and mannose α1,3 branches respectively.

Tetra-antennary N-linked oligosachharides most commonly provide four possible sites for sialic acid attachment while bi- and tri-antennary oligosaccharide chains, which can substitute for the tetra-antennary form at Asn-liked sites, commonly have at most only two or three sialic acids attached. O-linked oligosaccharides commonly provide only two sites for sialic acid attachement. Mammalian cell cultures can be screened for those cells that preferentially add teta-antennary chains to the G-CSF analogs of the present invention, thereby maximizing the number of sites for sialic acid attachment. Different types of mammalian cells also differ with respect to the transferase enzymes present and consequently the sialic acid content and type of oligosachharide attached at each site. One way to optimize the carbohydrate content for a given G-CSF analog is to express the analog in a cell line wherein an expression plasmid containing DNA encoding a specific sialyl transferase (e.g., α2,6 sialyltrasnferase) is co-transfected with the G-CSF analog expression plasmid. Alternatively a host cell line may be stably transfected with a sialyl transferase cDNA and that host cell used to express the G-CSF analog of interest. Thus, it is preferable if the oligosaccharide structure and sialic acid content are optimized for each analog encompassed by the present invention.

For purposes of the present invention, as disclosed and claimed herein, the following terms and abbreviations are defined below. The terms and abbreviations used in this document have their normal meanings unless otherwise designated. For example, “° C.” refers to degrees Celsius; “mmol” refers to millimole or millimoles; “mg” refers to milligrams; “μg” refers to micrograms; “ml or mL” refers to milliliters; and “μl or μL” refers to microliters. Amino acids abbreviations are as set forth in 37 C.F.R. § 1.822 (b)(2) (1994).

“Granulocyte-colony stimulating factor” means human granulocyte colony stimuating factor, and is abbreviated herein as “G-CSF.” G-CSF is a four helix bundle cytokine that supports growth of guanulocyte colonies in vitro and stimulates granulopoiesis in vivo. The amino acid sequence of G-CSF is known. The predominant form of G-CSF consists of 174 amino acids and has an O-linked carbohydrate at position 133.

“G-CSF analog” refers to human G-CSF with one or more changes in the amino acid sequence which result in an increase in the number of sites for carbohydrate attachment compared with native human G-CSF expressed in animal cells in vivo. G-CSF analog also refers to human G-CSF wherein the O-linked glycosylation site at position 133 is replaced with a N-linked glycosylation site.

“G-CSF activity” refers to the ability of a compound to stimulate granulopoiesis. Granulopoietic activity can be assessed in vitro as well as in vivo. Granulopoietic activity generally refers to the ability of a compound to cause an increase in the number of circulating neutrophils from an established baseline when administered by an acceptable route of administration at effective doses. In vitro activity can be determined by the method outlined in Example 6 and in vivo activity can be determined by the method outlined in Example 7.

“Neupogen®” refers to commercially available human granulocyte-colony stimulating factor (G-CSF), produced by recombinant DNA technology. Neupogen® is the Amgen, Inc. trademark for Filgrastim®, which has been selected as the name for recombinant methionyl human granulocyte colony-stimulating factor (r-metHuG-CSF). Neupogen® is a 175 amino acid protein having a molecular weight of 18,800 daltons and is produced by E coli. The amino acid sequence is identical to the natural G-CSF sequence, except for the addition of an N-terminal methionine necessary for expression in E coli.

Preferred G-CSF analogs are defined below. While these analogs are defined as having Ala at position 17 instead of Cys, a person of ordinary skill in the art would understand that analogs with the natural amino acid at position 17 or additional substitutions such as Leu, Glu, or Ser at position 17 are also included in the scope of these preferred analogs. The Cys at position 17 does not appear important for activity; however, the presence of a free thiol group when Cys is present can induce aggregation.

Preferred G-CSF analogs with granulopoietic activity include the following: G-CSF[A37N,Y39T] which is a protein of Formula I wherein:

Xaa at position 17 is Ala;

Xaa at position 37 is Asn;

Xaa at position 38 is Thr;

Xaa at position 39 is Thr;

Xaa at position 57 is Pro;

Xaa at position 58 is Trp;

Xaa at position 59 is Ala;

Xaa at position 60 is Pro;

Xaa at position 61 is Leu;

Xaa at position 62 is Ser;

Xaa at position 63 is Ser;

Xaa at position 64 is Cys;

Xaa at position 65 is Pro;

Xaa at position 66 is Ser;

Xaa at position 67 is Gln;

Xaa at position 68 is Ala;

Xaa at position 69 is Leu;

Xaa at position 93 is Glu;

Xaa at position 94 is Gly;

Xaa at position 95 is Ile;

Xaa at position 97 is Pro;

Xaa at position 133 is Thr;

Xaa at position 134 is Gln;

Xaa at position 135 is Gly;

Xaa at position 141 is Ala;

Xaa at position 142 is Ser; and

Xaa at position 143 is Ala. [SEQ ID NO:17]

G-CSF[P57V,W58N,P60T] which is a protein of Formula I wherein:

Xaa at position 17 is Ala;

Xaa at position 37 is Ala;

Xaa at position 38 is Thr;

Xaa at position 39 is Tyr;

Xaa at position 57 is Val;

Xaa at position 58 is Asn;

Xaa at position 59 is Ala;

Xaa at position 60 is Thr;

Xaa at position 61 is Leu;

Xaa at position 62 is Ser;

Xaa at position 63 is Ser;

Xaa at position 64 is Cys;

Xaa at position 66 is Ser;

Xaa at position 65 is Pro;

Xaa at position 67 is Gln;

Xaa at position 68 is Ala;

Xaa at position 69 is Leu;

Xaa at position 93 is Glu;

Xaa at position 94 is Gly;

Xaa at position 95 is Ile;

Xaa at position 97 is Pro;

Xaa at position 133 is Thr;

Xaa at position 134 is Gln;

Xaa at position 135 is Gly;

Xaa at position 141 is Ala;

Xaa at position 142 is Ser; and

Xaa at position 143 is Ala. [SEQ ID NO:18]

G-CSF[P60N,S62T] which is a protein of Formula I wherein:

Xaa at position 17 is Ala;

Xaa at position 37 is Ala;

Xaa at position 38 is Thr;

Xaa at position 39 is Tyr;

Xaa at position 57 is Pro;

Xaa at position 58 is Trp;

Xaa at position 59 is Ala;

Xaa at position 60 is Asn;

Xaa at position 61 is Leu;

Xaa at position 62 is Thr;

Xaa at position 63 is Ser;

Xaa at position 64 is Cys;

Xaa at position 65 is Pro;

Xaa at position 66 is Ser;

Xaa at position 67 is Gln;

Xaa at position 68 is Ala;

Xaa at position 69 is Leu;

Xaa at position 93 is Glu;

Xaa at position 94 is Gly;

Xaa at position 95 is Ile;

Xaa at position 97 is Pro;

Xaa at position 133 is Thr;

Xaa at position 134 is Gln;

Xaa at position 135 is Gly;

Xaa at position 141 is Ala;

Xaa at position 142 is Ser; and

Xaa at position 143 is Ala. [SEQ ID NO:19]

G-CSF[S63N,P65T] which is a protein of Formula I wherein:

Xaa at position 17 is Ala;

Xaa at position 37 is Ala;

Xaa at position 38 is Thr;

Xaa at position 39 is Tyr;

Xaa at position 57 is Pro;

Xaa at position 58 is Trp;

Xaa at position 59 is Ala;

Xaa at position 60 is Pro;

Xaa at position 61 is Leu;

Xaa at position 62 is Ser;

Xaa at position 63 is Asn;

Xaa at position 64 is Cys;

Xaa at position 65 is Thr;

Xaa at position 66 is Ser;

Xaa at position 67 is Gln;

Xaa at position 68 is Ala;

Xaa at position 69 is Leu;

Xaa at position 93 is Glu;

Xaa at position 94 is Gly;

Xaa at position 95 is Ile;

Xaa at position 97 is Pro;

Xaa at position 133 is Thr;

Xaa at position 134 is Gln;

Xaa at position 135 is Gly;

Xaa at position 141 is Ala;

Xaa at position 142 is Ser; and

Xaa at position 143 is Ala. [SEQ ID NO:20]

G-CSF[Q67N,L69T] which is a protein of Formula I wherein:

Xaa at position 17 is Ala;

Xaa at position 37 is Ala;

Xaa at position 38 is Thr;

Xaa at position 39 is Tyr;

Xaa at position 57 is Pro;

Xaa at position 58 is Trp;

Xaa at position 59 is Ala;

Xaa at position 60 is Pro;

Xaa at position 61 is Leu;

Xaa at position 62 is Ser;

Xaa at position 63 is Ser;

Xaa at position 64 is Cys;

Xaa at position 65 is Pro;

Xaa at position 66 is Ser;

Xaa at position 67 is Asn;

Xaa at position 68 is Ala;

Xaa at position 69 is Thr;

Xaa at position 93 is Glu;

Xaa at position 94 is Gly;

Xaa at position 95 is Ile;

Xaa at position 97 is Pro;

Xaa at position 133 is Thr;

Xaa at position 134 is Gln;

Xaa at position 135 is Gly;

Xaa at position 141 is Ala;

Xaa at position 142 is Ser; and

Xaa at position 143 is Ala. [SEQ ID NO:21]

G-CSF[E93N,I95T] which is a protein of Formula I wherein:

Xaa at position 17 is Ala;

Xaa at position 37 is Ala;

Xaa at position 38 is Thr;

Xaa at position 39 is Tyr;

Xaa at position 57 is Pro;

Xaa at position 58 is Trp;

Xaa at position 59 is Ala;

Xaa at position 60 is Pro;

Xaa at position 61 is Leu;

Xaa at position 62 is Ser;

Xaa at position 63 is Ser;

Xaa at position 64 is Cys;

Xaa at position 65 is Pro;

Xaa at position 66 is Ser;

Xaa at position 67 is Gln;

Xaa at position 68 is Ala;

Xaa at position 69 is Leu;

Xaa at position 93 is Asn;

Xaa at position 94 is Gly;

Xaa at position 95 is Thr;

Xaa at position 97 is Pro;

Xaa at position 133 is Thr;

Xaa at position 134 is Gln;

Xaa at position 135 is Gly;

Xaa at position 141 is Ala;

Xaa at position 142 is Ser; and

Xaa at position 143 is Ala. [SEQ ID NO:22]

G-CSF[T133N,G135T] which is a protein of Formula I wherein:

Xaa at position 17 is Ala;

Xaa at position 37 is Ala;

Xaa at position 38 is Thr;

Xaa at position 39 is Tyr;

Xaa at position 57 is Pro;

Xaa at position 58 is Trp;

Xaa at position 59 is Ala;

Xaa at position 60 is Pro;

Xaa at position 61 is Leu;

Xaa at position 62 is Ser;

Xaa at position 63 is Ser;

Xaa at position 64 is Cys;

Xaa at position 65 is Pro;

Xaa at position 66 is Ser;

Xaa at position 67 is Gln;

Xaa at position 68 is Ala;

Xaa at position 69 is Leu;

Xaa at position 93 is Glu;

Xaa at position 94 is Gly;

Xaa at position 95 is Ile;

Xaa at position 97 is Pro;

Xaa at position 133 is Asn;

Xaa at position 134 is Gln;

Xaa at position 135 is Thr;

Xaa at position 141 is Ala;

Xaa at position 142 is Ser; and

Xaa at position 143 is Ala. [SEQ ID NO:23]

G-CSF[A141N,A143T] which is a protein of Formula I wherein:

Xaa at position 17 is Ala;

Xaa at position 37 is Ala;

Xaa at position 38 is Thr;

Xaa at position 39 is Tyr;

Xaa at position 57 is Pro;

Xaa at position 58 is Trp;

Xaa at position 59 is Ala;

Xaa at position 60 is Pro;

Xaa at position 61 is Leu;

Xaa at position 62 is Ser;

Xaa at position 63 is Ser;

Xaa at position 64 is Cys;

Xaa at position 65 is Pro;

Xaa at position 66 is Ser;

Xaa at position 67 is Gln;

Xaa at position 68 is Ala;

Xaa at position 69 is Leu;

Xaa at position 93 is Glu;

Xaa at position 94 is Gly;

Xaa at position 95 is Ile;

Xaa at position 97 is Pro;

Xaa at position 133 is Thr;

Xaa at position 134 is Gln;

Xaa at position 135 is Gly;

Xaa at position 141 is Asn;

Xaa at position 142 is Ser; and

Xaa at position 143 is Thr. [SEQ ID NO:24]

G-CSF[A37N,Y39T,T133N,G135T] which is G-CSF[A37N,Y39T] wherein Xaa at position 133 is Asn and Xaa at position 135 is Thr.

G-CSF[A37N,Y39T,A141N,A143T] which is G-CSF[A37N,Y39T] wherein Xaa at position 141 is Asn and Xaa at position 143 is Thr.

G-CSF[A37N,Y39T,P57V,W58N,P60T] which is G-CSF[A37N,Y39T] wherein Xaa at position 57 is Val, Xaa at position 58 is Asn and Xaa at position 60 is Thr.

G-CSF[A37N,Y39T,P60N,S62T] which is G-CSF[A37N,Y39T] wherein Xaa at position 60 is Asn and Xaa at position 62 is Thr.

G-CSF[A37N,Y39T,S63N,P65T] which is G-CSF[A37N,Y39T] wherein Xaa at position 63 is Asn and Xaa at postion 65 is Thr.

G-CSF[A37N,Y39T,Q67N,L69T] which is G-CSF[A37N,Y39T] wherein Xaa at position 67 is Asn and Xaa at position 69 is Thr.

G-CSF[A37N,Y39T,E93N,I95T] which is G-CSF[A37N,Y39T] wherein Xaa at position 93 is Asn and Xaa at position 95 is Thr.

G-CSF[A37N,Y39T,P57V,W58N,P60T,S63N,P65T] which is G-CSF[A37N,Y39T] wherein Xaa at position 57 is Val, Xaa at position 58 is Asn, Xaa at position 60 is Thr, Xaa at position 63 is Asn, and Xaa at position 65 is Thr.

G-CSF[A37N,Y39T,P57V,W58N,P60T,Q67N,L69T] which is G-CSF[A37N,Y39T] wherein Xaa at position 57 is Val, Xaa at position 58 is Asn, Xaa at position 60 is Thr, Xaa at position 67 is Asn and Xaa at position 69 is Thr.

G-CSF[A37N,Y39T,S63N,P65T,E93N,I95T] which is G-CSF[A37N,Y39T] wherein Xaa at position 63 is Asn, Xaa at position 65 is Thr, Xaa at position 93 is Asn, and Xaa at position 95 is Thr.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids such as norleucine, β-alanine, ornithine, GABA, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The incorporation of non-natural amino acids, including synthetic non-native amino acids, substituted amino acids, or one or more D-amino acids into the G-CSF analogs of the present invention can be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore less likely to induce humoral immune responses in the whole organism.

Native G-CSF can be used as the backbone to create the glycosylated G-CSF analogs of the present invention. In addition, the native G-CSF backbone used to create the analogs of the present invention can be modified such that substitutions in the regions defined in FIG. 1 are made in the context of a different or improved G-CSF protein. For example, native G-CSF with a Cystein to Alanine substitution at position 17 may reduce aggregation and enhance stability and thus, can be used as the backbone used to create the glycosylated G-CSF analogs of the present invention.

In addition, Reidhaar-Olson et al., through alanine scanning mutagenesis, describe residues critical to the activity of human G-CSF. [Reidhaar-Olson et al. (1996) Biochemistry 35:9034-9041; See also Young et al. (1997) Protein Science 6:1228-1236]. Thus, the glycosylated analogs of the present invention can be modified by substituting amino acids outside the glycosylated regions described in FIG. 1.

In addition to published structure/function analyses such as the alanine scanning studies described above, there are numerous factors that can be considered when selecting amino acids for substitution in the glycosylated G-CSF analog described herein. One factor that can be considered in making such changes is the hydropathic index of amino acids. The importance of the hydropathic amino acid index in conferring interactive biological function on a protein has been discussed by Kyte and Doolittle (1982, J. Mol. Biol., 157: 105-132). It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide, polypeptide, or protein can be substituted for other amino acids having a similar hydropathic index or score and produce a resultant peptide, etc., having similar or even improved biological activity. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5+1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within ±1 are more preferred, and those within ±0.5 are most preferred.

As outlined above, amino acid substitutions in the glycsolated G-CSF analogs of the present invention can be based on the relative similarity of the amino acid side-chain substituents, for example, their hydrophobicity, hydrophilicity, charge, size, etc. Furthermore, substitutions can be made based on secondary structure propensity. For example, a helical amino acid can be replaced with an amino acid that would preserve the helical structure. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine.

“Base pair” or “bp” as used herein refers to DNA or RNA. The abbreviations A, C, G, and T correspond to the 5′-monophosphate forms of the deoxyribonucleosides (deoxy)adenosine, (deoxy)cytidine, (deoxy)guanosine, and thymidine, respectively, when they occur in DNA molecules. The abbreviations U, C, G, and A correspond to the 5′-monophosphate forms of the ribonucleosides uridine, cytidine, guanosine, and adenosine, respectively when they occur in RNA molecules. In double stranded DNA, base pair may refer to a partnership of A with T or C with G. In a DNA/RNA, heteroduplex base pair may refer to a partnership of A with U or C with G. (See the definition of “complementary”, infra.)

“Digestion” or “Restriction” of DNA refers to the catalytic cleavage of the DNA with a restriction enzyme that acts only at certain sequences in the DNA (“sequence-specific endonucleases”). The various restriction enzymes used herein are commercially available and their reaction conditions, cofactors, and other requirements were used as would be known to one of ordinary skill in the art. Appropriate buffers and substrate amounts for particular restriction enzymes are specified by the manufacturer or can be readily found in the literature.

“Ligation” refers to the process of forming phosphodiester bonds between two double stranded nucleic acid fragments. Unless otherwise provided, ligation may be accomplished using known buffers and conditions with a DNA ligase, such as T4 DNA ligase.

“Plasmid” refers to an extrachromosomal (usually) self-replicating genetic element. Plasmids are generally designated by a lower case “p” followed by letters and/or numbers. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan.

“Recombinant DNA cloning vector” as used herein refers to any autonomously replicating agent, including, but not limited to, plasmids and phages, comprising a DNA molecule to which one or more additional DNA segments can or have been added.

“Recombinant DNA expression vector” as used herein refers to any recombinant DNA cloning vector in which a promoter to control transcription of the inserted DNA has been incorporated.

“Transcription” refers to the process whereby information contained in a nucleotide sequence of DNA is transferred to a complementary RNA sequence.

“Transfection” refers to the uptake of an expression vector by a host cell whether or not any coding sequences are, in fact, expressed. Numerous methods of transfection are known to the ordinarily skilled artisan, for example, calcium phosphate co-precipitation, liposome transfection, and electroporation. Successful transfection is generally recognized when any indication of the operation of this vector occurs within the host cell.

“Transformation” refers to the introduction of DNA into an organism so that the DNA is replicable, either as an extrachromosomal element or by chromosomal integration. Methods of transforming bacterial and eukaryotic hosts are well known in the art, many of which methods, such as nuclear injection, protoplast fusion or by calcium treatment using calcium chloride are summarized in J. Sambrook, et al., Molecular Cloning: A Laboratory Manual, (1989). Generally, when introducing DNA into Yeast the term transformation is used as opposed to the term transfection.

“Translation” as used herein refers to the process whereby the genetic information of messenger RNA (mRNA) is used to specify and direct the synthesis of a polypeptide chain.

“Vector” refers to a nucleic acid compound used for the transfection and/or transformation of cells in gene manipulation bearing polynucleotide sequences corresponding to appropriate protein molecules which, when combined with appropriate control sequences, confers specific properties on the host cell to be transfected and/or transformed. Plasmids, viruses, and bacteriophage are suitable vectors. Artificial vectors are constructed by cutting and joining DNA molecules from different sources using restriction enzymes and ligases. The term “vector” as used herein includes Recombinant DNA cloning vectors and Recombinant DNA expression vectors.

“Complementary” or “Complementarity”, as used herein, refers to pairs of bases (purines and pyrimidines) that associate through hydrogen bonding in a double stranded nucleic acid. The following base pairs are complementary: guanine and cytosine; adenine and thymine; and adenine and uracil.

“Hybridization” as used herein refers to a process in which a strand of nucleic acid joins with a complementary strand through base pairing. The conditions employed in the hybridization of two non-identical, but very similar, complementary nucleic acids varies with the degree of complementarity of the two strands and the length of the strands. Such techniques and conditions are well known to practitioners in this field.

“Isolated amino acid sequence” refers to any amino acid sequence, however, constructed or synthesized, which is locationally distinct from the naturally occurring sequence.

“Isolated DNA compound” refers to any DNA sequence, however constructed or synthesized, which is locationally distinct from its natural location in genomic DNA.

“Isolated nucleic acid compound” refers to any RNA or DNA sequence, however constructed or synthesized, which is locationally distinct from its natural location.

“Primer” refers to a nucleic acid fragment which functions as an initiating substrate for enzymatic or synthetic elongation.

“Promoter” refers to a DNA sequence which directs transcription of DNA to RNA.

“Probe” refers to a nucleic acid compound or a fragment, thereof, which hybridizes with another nucleic acid compound.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while short probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature that can be used. As a result, it follows that higher relative temperatures would tend to make the reactions more stringent, while lower temperatures less so. For additional details and explanation of stringency of hybridization reactions, see Ausubel et al., Current Protocols in Molecular Biology, Wiley Interscience Publishers, 1995.

“Stringent conditions” or “high stringency conditions”, as defined herein, may be identified by those that (1) employ low ionic strength and high temperature for washing, for example, 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% ficoll/0.1% polyvinylpyrrolidone/50 mM sodium phosphate buffer at pH 6.5 with 750 mM sodium chloride/75 mM sodium citrate at 42° C.; or (3) employ 50% formamide, 5×SSC (750 mM sodium chloride, 75 mM sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C. with washes at 42° C. in 0.2×SSC (30 mM sodium chloride/3 mM sodium citrate) and 50% formamide at 55° C., followed by a high-stringency wash consisting of 0.1×SSC containing EDTA at 55° C.

“Moderately stringent conditions” may be identified as described by Sambrook et al. [Molecular Cloning: A Laboratory Manual, New York: Cold Spring Harbor Press, (1989)], and include the use of washing solution and hybridization conditions (e.g., temperature, ionic strength, and % SDS) less stringent than those described above. An example of moderately stringent conditions is overnight incubation at 37° C. in a solution comprising: 20% formamide, 5×SSC (750 mM sodium chloride, 75 mM sodium citrate), 50 mM sodium phosphate at pH 7.6, 5× Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc., as necessary to accommodate factors such as probe length and the like.

“PCR” refers to the widely-known polymerase chain reaction employing a thermally-stable DNA polymerase.

“Leader sequence” refers to a sequence of amino acids which can be enzymatically or chemically removed to produce the desired polypeptide of interest.

“Secretion signal sequence” refers to a sequence of amino acids generally present at the N-terminal region of a larger polypeptide functioning to initiate association of that polypeptide with the cell membrane and secretion of that polypeptide through the cell membrane.

DNA encoding human G-CSF can be obtained from a cDNA library prepared from tissue or cells which express G-CSF mRNA at a detectable level such as monocytes, macrophages, vascular endothelial cells, fibroblasts, and some human malignant and leukemic myeloblastic cells. Libraries can be screened with probes designed using the published DNA sequence for human G-CSF. [Souza L. et al. (1986) Science 232:61-65]. Screening a cDNA or genomic library with the selected probe may be conducted using standard procedures, such as described in Sambrook et al., Molecular Cloning: A Laboratory Manual, Cold Spring Harbor Laboratory Press, NY (1989). An alternative means to isolate the gene encoding human G-CSF is to use PCR methodology [Sambrook et al., supra; Dieffenbach et al., PCR Primer: A Laboratory Manual, Cold Spring Harbor Laboratory Press, NY (1995)].

The glycosylated G-CSF analogs of the present invention can be constructed by a variety of mutagenesis techniques well known in the art. Specifically, a representative number of glycosylated G-CSF analogs were constructed using mutagenic PCR from a cloned wild-type human G-CSF DNA template (Example 1). The mutagenic PCR method utilizes strand overlap extension to create specific base mutations for the purposes of changing a specific amino acid sequence (FIG. 2) in the corresponding protein. The primers were also designed to create a restriction enzyme site to facilitate screening of positive clones.

This PCR mutagenesis requires the use of four primers, two in the forward orientation (primers A and C, FIG. 2) and two in the reverse orientation (primers B and D, FIG. 2). A mutated gene is amplified from the wild-type template in two different stages. The first reaction amplifies the gene in halves by performing an A to B reaction and a separate C to D reaction wherein the B and C primers target the area of the gene to be mutated. When aligning these primers with the target area, they contain mismatches for the bases that are targeted to be changed. Once the A to B and C to D reactions are complete, the reaction products are isolated and mixed for use as the template for the A to D reaction. This reaction then yields the full, mutated product. PCR mutagenesis was used to make a representative number of polynucelotides encoding G-CSF analogs that have consensus N-linked glycosylation sites in one or more regions as defined in FIG. 1 (Example 1).

The glycosylated G-CSF analogs of the present invention may be produced by a variety of methods including recombinant DNA technology or well known chemical procedures, such as solution or solid-phase peptide synthesis, or semi-synthesis in solution beginning with protein fragments coupled through conventional solution methods.

Recombinant DNA methods are preferred for producing the glycosylated G-CSF analogs of the present invention. Host cells are transfected or transformed with expression or cloning vectors described herein for glycosylated G-CSF analog production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra. Methods of transfection are known to the ordinarily skilled artisan, for example, CaPO₄, lisposome transfection, and electroporation. General aspects of mammalian cell host system transformations have been described in U.S. Pat. No. 4,399,216. For various techniques for transforming mammalian cells, see Keown et al., Methods in Enzymology 185: 527-37 (1990) and Mansour et al., Nature 336(6197): 348-52 (1988).

Suitable host cells for cloning or expressing the nucleic acid (e.g., DNA) in the vectors herein include mammalian cells having the appropriate endogenous enzymes to glycosylate the analogs of the present invention. These are generally cells derived from multicellular organisms. Examples of invertebrate cells include insect cells such as Drosophila S2 and Spodoptera Sp, Spodoptera high5 as well as plant cells. Examples of useful mammalian host cell lines include Chinese hamster ovary (CHO) and COS cells. More specific examples include monkey kidney CVl line transformed by SV40 (COS-7, ATCC CRL 1651); human embryonic kidney line [293T or 293/EBNA cells subcloned for growth in suspension culture, Graham et al., J. Gen Virol., 36(1): 59-74 (1977)]; Chinese hamster ovary cells/-DHFR [CHO, Urlaub and Chasin, Proc. Natl. Acad. Sci. USA, 77(7): 4216-20 (1980)]; CHO-K1; CHO-DG44; mouse sertoli cells [TM4, Mather, Biol. Reprod. 23(1):243-52 (1980)]; human lung cells (W138. ATCC CCL 75); human liver cells (Hep G2, HB 8065); and mouse mammary tumor (MMT 060562, ATCC CCL51). The selection of the appropriate host cell is deemed to be within the skill in the art.

Glycosylated G-CSF analogs may be produced recombinantly not only directly, but also as a fusion polypeptide with a heterologous polypeptide, which may be a signal sequence or other polypeptide having a specific cleavage site at the N-terminus of the mature protein or polypeptide. The signal sequence may be one from another glycosylated protein. For example, G-CSF[A37N,Y39T,P57Y,W58N,P60T,L69T] was successfully expressed using both the erythropoietin leader sequence as well as the endogenous G-CSF leader sequence. In general, the signal sequence may be a component of the vector, or it may be a part of the G-CSF analog-encoding DNA that is inserted into the vector. A mammalian signal sequence may be used to direct secretion of the protein, such as signal sequences from various secreted polypeptides as well as viral secretory leaders.

Both expression and cloning vectors contain a nucleic acid sequence that enables the vector to replicate in one or more selected host cells. Expression and cloning vectors will typically contain a selection gene, also termed a selectable marker. An example of suitable selectable markers for mammalian cells are those that enable the identification of cells competent to take up the G-CSF analog-encoding nucleic acid, such as DHFR, thymidine kinase, or markers providing the cell with neomycin or puromycin resistance. An appropriate host cell when wild-type DHFR is employed is the CHO cell line deficient in DHFR activity, prepared and propagated as described by Urlaub and Chasin, (1980) Proc. Natl. Acad. Sci. USA, 77: 4216-20.

Expression and cloning vectors usually contain a promoter operably linked to the G-CSF analog-encoding nucleic acid sequence to direct mRNA synthesis. Promoters recognized by a variety of potential host cells are well known. Transcription of mRNA from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a polynucleotide encoding a G-CSF analog may be increased by inserting an enhancer sequence into the vector. Enhancers are cis-acting elements of DNA, usually about from 10 to 300 bp, that act on a promoter to increase its transcription. Many enhancer sequences are now known from mammalian genes (e.g., globin, elastase, albumin, α-ketoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the G-CSF analog coding sequence, but is preferably located at a site 5′ from the promoter.

Expression vectors used in eukaryotic host cells will also contain sequences necessary for the termination of transcription and for stabilizing the mRNA. Such sequences are commonly available from the 5′ and occasionally 3′ untranslated regions of eukaryotic or viral DNAs or cDNAs. These regions contain nucleotide segments transcribed as polyadenylated fragments in the untranslated portion of the mRNA.

Once the glycosylated G-CSF analogs of the present invention are expressed in the appropriate host cell, the analogs can be isolated and purified. The following procedures are exemplary of suitable purification procedures: fractionation on carboxymethyl cellulose; gel filtration such as Sephadex G-75; anion exchange resin such as DEAE or Mono-Q; cation exchange such as CM or Mono-S; protein A sepharose to remove contaminants such as IgG; metal chelating columns to bind epitope-tagged forms of the polypeptide; reversed-phase HPLC; chromatofocusing; silica gel; ethanol precipitation; and ammonium sulfate precipitation.

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend, for example, on the nature of the production process used and the particular glycosylated analog produced. Specifically, a representative number of glycosylated G-CSF analogs were purified as described in Example 3.

Several analytical methods exist to identify patterns of glycosylation. These methods include: SDS-PAGE wherein the differential migration of proteins can be identified using Coomassie blue staining or immunoblotting using anti-GCSF antibodies, lectin blots, matrix assisted laser desorption/ionization-mass spectrometry (MALDI-MS), liquid chromatography/mass spectrometry (LC/MS) of protein fragments, oligoprofiling and complete oligosaccharide analysis by LC/MS or high-pH anion exchange chromatography with pulsed amperometric detection (HPAE-PAD). Isoelectric focusing, analytical anion exchange, chromatofocussing, and capillary electrophoresis can all be used to identify extent of sialylation and to compare glycoforms of any one glycosylation site. A representative number of glycosylated G-CSF analogs were purified and characterized using some of the above methodology (see Examples 4 and 5).

For example, Table 2 illustrates the molecular mass of various glycosylated G-CSF analogs as determined by MALDI-MS. In contrast to the non-glycosylated Neupogen®, which displays a sharp peak, the G-CSF analogs display broad peaks. These broad peaks indicate the presence of glycosylation on the compounds. Immunoblots showed that these peaks represented G-CSF analogs (See Example 5).

To provide additional confirmation that the analogs were being secreted and glycosylated in the host cells being employed, SDS-PAGE followed by immunoblotting was performed. A representative number of glycosylated G-CSF analogs are shown in FIG. 6. Glycosylated analogs such as G-CSF[A37N, Y39T], G-CSF[P57N,W58N,P60T], G-CSF[A37N,Y39T,P57V,W58N, P60T], and G-CSF[A37N,Y39T,L69T] all migrated more slowly than Neupogen® or wild-type G-CSF expressed in CHO-K1 cells indicating the presence of increased carbohydrate content. Analogs with more than one region glycosylated migrated more slowly than those with a single region glycosylated.

A reprentative number of glycosylated G-CSF analogs were also tested for activity. Numerous methods exist to detect G-CSF-like activity. One such method employs bone marrow cells from mouse femurs. G-CSF or analogs thereof can be incubated with cultured cells and subsequent cell growth can be quantitated by thymidine incorporation or calorimetrically at the end of the assay period. [See also Holmes et al., (1985), Proc. Natl. Acad. Sci., 82:6687-6691; Nicholson et al., (1994) Proc. Natl. Acad. Sci. 91:2985-2988; Yamaguchi et al. (1997) Biol. Pharm. Bull. 20:943-947; Kuga T. et al. (1989) Biochem. Biophys. Res. Comm. 159:103-111].

Specifically, a representative number of glycosylated G-CSF analogs were tested using cells that express the G-CSF receptor (see Example 6). Cells were stably transfected with a plasmid containing a reporter gene under the control of a STAT-binding sequence which can accommodate all STAT family members. Purified glycosylated G-CSF analogs were incubated with these cells to determine if the analogs could bind the G-CSF receptor and transduce a signal that would ultimately be measured as reporter expression. Table 2 provides EC₅₀ values relative to that obtained for Neupogen®. The EC₅₀ is the concentration of protein giving 50% maximal reporter activity (Example 6).

The glycosylated G-CSF analogs tested had varying degrees of activity compared with Neupogen®. Interestingly, the triple glycosylated G-CSF analog had in vitro activity indistinguishable from Neupogen®. This activity data suggests that the glycosylated G-CSF analogs of the present invention retain the proper three-dimensional structure to activate the G-CSF receptor.

Physical stability is an essential feature for therapeutic formulations. The physical stability of the glycosylated G-CSF analogs of the present invention depends on their conformational stability, the number of charged residues (pI of the protein), the ionic strength and pH of the formulation, and the protein concentration, among other possible factors. As discussed previously, the G-CSF analogs of the present invention can be successfully glycosylated and expressed such that they maintain their three dimensional structure. Because these analogs are able to fold properly in a hyperglycosylated state, they will have improved conformational and physical stability relative to wild-type G-CSF.

While wild-type G-CSF produced in mammalian cells and bacterial cells has similar activity in vivo, the mammalian cell-produced protein has increased conformational and physical stability due to the presence of a single O-linked sugar moiety present at position 133. Thus, the G-CSF analogs of the present invention, which have an increased glycosylation content compared to wild-type G-CSF produced in mammalian or bacterial cells, will have increased stability. Furthermore, it is likely that glycosylation may inhibit inter-domain interactions and consequently enhance stability by preventing inter-domain disulfide shuffling.

Example 8 illustrates a representative number of glycosylated G-CSF analogs which have increased stability compared to wild-type G-CSF expressed in CHO cells and G-CSF[C17A] expressed in 293 EBNA cells.

The present invention thus, provides glycosylated G-CSF analogs that have improved biochemical and biophysical properties. The G-CSF analogs of the present invention can be successfully expressed and glycosylated in mammalian cells and retain their three-dimensional structure and corresponding activity. The carbohydrate moieties present on these novel analogs will affect clearance mechanisms resulting in compounds with an extended plasma half-life compared to the wild-type G-CSF compounds currently on the market.

The following examples are presented to further describe the present invention. The scope of the present invention is not to be construed as merely consisting of the following examples. Those skilled in the art will recognize that the particular reagents, equipment, and procedures described are merely illustrative and are not intended to limit the present invention in any manner.

### EXAMPLE 1

### Construction of DNA Encoding Glycosylated G-CSF Analogs

Table 1 provides the sequence of primers used to create functional glycosylation sites in different regions of the protein (See FIG. 1).

TABLE 1

Primer sequences used to introduce mutations into
human G-CSF.

| | | | | |
|---|---|---|---|---|
| Mutation | A Primer* | B Primer* | C Primer* | D Primer* |

| | | | | |
|---|---|---|---|---|
| WT | CF177 [SEQ ID | CF178 [SEQ ID | CF179 [SEQ ID | CF176 [SEQ ID |
| | NO:25] | NO:26] | NO:27] | NO:28] |
| | [symbol] [symbol] [symbol] [symbol] [symbol] [symbol] [symbol] [symbol] [symbol] [symbol] [symbol] [symbol] | GGGGCAGGGAGC | GGACAGTGCAGG | [symbol] [symbol] [symbol] [symbol] [symbol] [symbol] [symbol] [symbol] [symbol] [symbol] [symbol] [symbol] |
| | [symbol] [symbol] [symbol] [symbol] [symbol] [symbol] [symbol] [symbol] [symbol] [symbol] [symbol] [symbol] | TGGCTGGGCCCA | AAGCCACTCCAC | [symbol] [symbol] [symbol] TCATTAGGG |
| | [symbol] [symbol] [symbol] [symbol] [symbol] [symbol] [symbol] [symbol] [symbol] ATG | GTGGAGTGGCTT | TGGGCCCAGCCA | CTGGGCAAGGTG |
| | GCCGGACCTGCC | CCTGCACTGTCC | GCTCCCTGCCCC | CCTTAAGACGCG |
| | ACCCAGAGCCCC | AGAGTGCACTGT | AGAGCTTCCTG | GTACGACACCTC |
| | ATGAAGCTG | G | | CAGGAAGCTCTG |
| C17A | CF177 [SEQ ID | C17Arev [SEQ | C17Afor [SEQ | CF176 [SEQ ID |
| SacI | NO:29] | ID NO:30] | ID NO:31] | NO:32] |
| | [symbol] [symbol] [symbol] [symbol] [symbol] [symbol] [symbol] [symbol] [symbol] [symbol] [symbol] [symbol] | GCTCTAAGGCCT | GGGCCCAGCGAG | [symbol] [symbol] [symbol] [symbol] [symbol] [symbol] [symbol] [symbol] [symbol] [symbol] [symbol] [symbol] |
| | [symbol] [symbol] [symbol] [symbol] [symbol] [symbol] [symbol] [symbol] [symbol] [symbol] [symbol] [symbol] | TGAGCAGGAAGC | CTCCCTGCCCCA | [symbol] [symbol] [symbol] TCATTAGGG |
| | [symbol] [symbol] [symbol] [symbol] [symbol] [symbol] [symbol] [symbol] [symbol] ATG | TCTGGGGCAGGG | GAGCTTCCTGCT | CTGGGCAAGGTG |
| | GCCGGACCTGCC | AGCTCGCTGGGC | CAAGGCCTTAGA | CCTTAAGACGCG |
| | ACCCAGAGCCCC | CCAGTGGAG | GCAAG | GTACGACACCTC |
| | ATGAAGCTG | | | CAGGAAGCTCTG |
| A37N, Y39T | CF177 [SEQ ID | A37Nrev [SEQ | A37Nfor [SEQ | CF176 [SEQ ID |
| SpeI | NO:33] | ID NO:34] | ID NO:35] | NO:36] |
| | [symbol] [symbol] [symbol] [symbol] [symbol] [symbol] [symbol] [symbol] [symbol] [symbol] [symbol] [symbol] | GTCCGAGCAGCA | GGCGCAGCGCTC | [symbol] [symbol] [symbol] [symbol] [symbol] [symbol] [symbol] [symbol] [symbol] [symbol] [symbol] [symbol] |
| | [symbol] [symbol] [symbol] [symbol] [symbol] [symbol] [symbol] [symbol] [symbol] [symbol] [symbol] [symbol] | CTAGTTCCTCGG | CAGGAGAAGCTG | [symbol] [symbol] [symbol] TCATTAGGG |
| | [symbol] [symbol] [symbol] [symbol] [symbol] [symbol] [symbol] [symbol] [symbol] ATG | GGTGGCACAGCT | TGTAACACCACC | CTGGGCAAGGTG |
| | GCCGGACCTGCC | TGGTGGTGTTAC | AAGCTGTGCCAC | CCTTAAGACGCG |
| | ACCCAGAGCCCC | ACAGCTTCTCCT | CCCGAGGAACTA | GTACGACACCTC |
| | ATGAAGCTG | G | GTGCTG | CAGGAAGCTCTG |
| T133N, | CF177 [SEQ ID | T133Nrev [SEQ | T133Nfor [SEQ | CF176 [SEQ ID |
| G135T | NO:37] | ID NO:38] | ID NO:39] | NO:40] |
| Eco47III | [symbol] [symbol] [symbol] [symbol] [symbol] [symbol] [symbol] [symbol] [symbol] [symbol] [symbol] [symbol] | GCCCGGCGCTGG | GGCCCCTGCCCT | [symbol] [symbol] [symbol] [symbol] [symbol] [symbol] [symbol] [symbol] [symbol] [symbol] [symbol] [symbol] |
| | [symbol] [symbol] [symbol] [symbol] [symbol] [symbol] [symbol] [symbol] [symbol] [symbol] [symbol] [symbol] | AAAGCGCTGGCG | GCAGCCCAACCA | [symbol] [symbol] [symbol] TCATTAGGG |
| | [symbol] [symbol] [symbol] [symbol] [symbol] [symbol] [symbol] [symbol] [symbol] ATG | AAGGCCGGCATG | GACCGCCATGCC | CTGGGCAAGGTG |
| | GCCGGACCTGCC | GCGGTCTGGTTG | GGCCTTCGCCAG | CCTTAAGACGCG |
| | ACCCAGAGCCCC | GGCTGCAGGGCA | CGCTTTCCAGCG | GTACGACACCTC |
| | ATGAAGCTG | G | | CAGGAAGCTCTG |
| A141N, | CF177 [SEQ ID | A141Nrev [SEQ | A141Nfor [SEQ | CF176 [SEQ ID |
| A143T | NO:41] | ID NO:42] | ID NO:43] | NO:44] |
| SapI | [symbol] [symbol] [symbol] [symbol] [symbol] [symbol] [symbol] [symbol] [symbol] [symbol] [symbol] [symbol] | GCCCGGCGCTGG | GGGAATGGCCCC | [symbol] [symbol] [symbol] [symbol] [symbol] [symbol] [symbol] [symbol] [symbol] [symbol] [symbol] [symbol] |
| | [symbol] [symbol] [symbol] [symbol] [symbol] [symbol] [symbol] [symbol] [symbol] [symbol] [symbol] [symbol] | AAGGTAGAGTTG | TGCTCTTCAGCC | [symbol] [symbol] [symbol] TCATTAGGG |
| | [symbol] [symbol] [symbol] [symbol] [symbol] [symbol] [symbol] [symbol] [symbol] ATG | AAGGCCGGCATG | CACCCAGGGTGC | CTGGGCAAGGTG |
| | GCCGGACCTGCC | GCACCCTGGGTG | CATGCCGGCCTT | CCTTAAGACGCG |
| | ACCCAGAGCCCC | GGCTGAAGAGCA | CAACTCTACCTT | GTACGACACCTC |
| | ATGAAGCTG | GGGGCCAT | CCAGCGCCGGGC | CAGGAAGCTCTG |
| | | | AG | |
| P57V, | JCB128 [SEQ | JCB136 [SEQ | JCB137 [SEQ | JCB129 [SEQ |
| W58N, P60T | ID NO:45] | ID NO:46] | ID NO:47] | ID NO:48] |
| HpaI | GCTAGCGGCGCG | GCTCAGGGTAGC | GGGCATCGTTAA | [symbol] [symbol] [symbol] TCGAGGATC |
| | CCACCATG | GTTAACGATGCC | CGCTACCCTGAG | CTCATTAGGGCT |
| | | CAGAGAGTG | CAGCTG | GGG |
| Q67N, L69T | JCB134 [SEQ | JCB138 [SEQ | JCB139 [SEQ | JCB135 [SEQ |
| NaeI | ID NO:49] | ID NO:50] | ID NO:51] | ID NO:52] |
| | GCTAGCGGCGCG | CAAGCAGCCGGC | GCCCCAGCAACG | [symbol] [symbol] [symbol] TCGAGGATC |
| | CCACCATGGCCG | CAGCTGGGTGGC | CCACCCAGCTGG | CTCATTAGGGCT |
| | GACCTGCCACCC | GTTGCTGGGGCA | CCGGCTGCTTGA | GGGCAAGGTGCC |
| | AG | GCTGCTCAG | G | TTAAGACGCGG |
| P60N, S62T | JCB128 [SEQ | JCB130 [SEQ | JCB131 [SEQ | JCB129 [SEQ |
| SpeI | ID NO:53] | ID NO:54] | ID NO:55] | ID NO:56] |
| | GCTAGCGGCGCG | GGGGCAACTAGT | GCTAACCTGACT | [symbol] [symbol] [symbol] TCGAGGATC |
| | CCACCATG | CAGGTTAGCCCA | AGTTGCCCCAGC | CTCATTAGGGCT |
| | | GGG | CAG | GGG |
| S63N, PG5T | JCB128 [SEQ | JCB132 [SEQ | JCB133 [SEQ | JCB129 [SEQ |
| MfeI | ID NO:57] | ID NO:58] | ID NO:59] | ID NO:60] |
| | GCTAGCGGCGCG | GGTGCAATTGCT | GCAATTGCACCA | [symbol] [symbol] [symbol] TCGAGGATC |
| | CCACCATG | CAGGGGAGCCCA | GCCAGGCCCTG | CTCATTAGGGCT |
| | | G | | GGG |
| E93N, I95T | JCB134 [SEQ | JCB140 [SEQ | JCB141 [SEQ | JCB135 [SEQ |
| BspEI | ID NO:61] | ID NO:62] | ID NO:63] | ID NO:64] |
| | GCTAGCGGCGCG | CCGGACTGGTCC | GAACGGGACCAG | [symbol] [symbol] [symbol] TCGAGGATC |
| | CCACCATGGCCG | CGTTCAGGGCCT | TCCGGAGTTGGG | CTCATTAGGGCT |
| | GACCTGCCACCC | GCAGGAGCCCCT | TCCCACCTTGG | GGGCAAGGTGCC |
| | AG | G | | TTAAGACGCGG |
| SalI | JCB155 [SEQ | | | |
| | ID NO:65] | | | |
| | [symbol] [symbol] [symbol] [symbol] [symbol] [symbol] GCTAGC | | | |
| | GGCGCGCCACCA | | | |
| | TGGCCGGACCTG | | | |

[symbol]

Nucleotides in bold represent changes imposed in the target sequence and nucleotides in bold and italics represent flanking sequences which may add restriction sites to facilitate cloning, Kozac sequences, or stop codons.

Preparation 1a: DNA Encoding Wild-Type Human G-CSF

A strand overlapping extension PCR reaction was used to create a wild type human G-CSF construct in order to eliminate the methylation of an ApaI site. Isolated human G-CSF cDNA served as the template for these reactions. The 5′ end A primer was used to create a restriction enzyme site prior to the start of the coding region as well as to introduce a Kozac sequence (GGCGCC) 5′ of the coding leader sequence to faciliate translation in cell culture.

The A-B product was generated using primers CF177 and CF178 in a PCR reaction. Likewise, the C-D product was produced with primers CF179 and CF176. The products were isolated and combined. The combined mixture was then used as a template with primers CF177 and CF178 to create the full-length wild-type construct. [Nelson, R. M. and Long, G. C. (1989), Anal. Biochem. 180:147-151].

The full-length product was ligated into the pCR2.1-Topo vector (Invitrogen, Inc. Cat. No. K4500-40) by way of a topoisomerase TA overhang system to create pCR2.1G-CSF.

The following protocol was used for preparation of the full-length wild-type G-CSF protein as well as each of the G-CSF analogs. Approximately 5 ng of template DNA and 15 pmol of each primer was used in the initial PCR reactions. The reactions were prepared using Platinum PCR Supermix® (GibcoBRL Cat. No; 11306-016). The PCR reactions were denatured at 94° C. for 5 min and then subject to 25 cycles wherein each cycle consisted of 30 seconds at 94° C. followed by 30 seconds at 60° C. followed by 30 seconds at 72° C. A final extension was carried out for 7 minutes at 72° C. PCR fragments were isolated from agarose gels and purified using a Qiaquick® gel extraction kit (Qiagen, Cat. No. #28706). DNA was resuspended in sterile water and used for the final PCR reaction to prepare full-length product.

Preparation 1b: DNA Encoding G-CSF[C17A] which is G-CSF Wherein the Amino Acid at Position 17 is Substituted with Ala was Constructed as Follows:

The wild-type construct in the pCR2.1-Topo vector (pCR2.1G-CSF) served as the PCR template for the C17A mutatgenesis. Strand ovelapping extension PCR was performed as described previously. CF177 and C17Arev served as the A-B primers and C17Afor and CF176 served as the C-D primers. The full-length mutated cDNA was prepared as described previously using the CF177 and CF176 primers. The B and C primers were used to mutate the DNA such that a SacI restriction site was created and the protein expressed from the full-length sequence contained an Alanine instead of a Cysteine at position 17. The full-length cDNA was ligated back into the pCR2.1-Topo vector to create pCR2.1G-CSF[C17A] wherein the sequence was confirmed. G-CSF analog encoding DNA was then cloned into the Nhe/Xho sites of mammalian expression vector pJB02 (FIG. 3) to create pJB02G-CSF[C17A].

Preparation 1c: DNA Encoding G-CSF[A37N,Y39T] was Constructed as Follows:

Strand overlapping extension PCR was performed using pCR2.1G-CSF[C17A] as the template. Primer CF177 and A37Nrev served as the A-B primers and CF176 and A37Nfor served as the C-D primers. The full-length mutated cDNA was prepared as described previously using the CF177 and CF176 primers. The B and C primers contained mismatched sequences such that a SpeI site was created in the DNA and the protein expressed from the full-length sequence contained a consensus sequence for N-linked glycosylation in region 1 of the protein (see Table 1, FIG. 1). The full-length cDNA was ligated back into the pCR2.1-Topo vector to create pCR2.1G-CSF[A37N,Y39T] wherein the sequence was confirmed. G-CSF analog encoding DNA was then cloned into the Nhe/Xho sites of mammalian expression vector pJB02 (FIG. 3) to create pJB02G-CSF[A37N,Y39T].

Preparation 1d: DNA Encoding G-CSF[P57V,W58N,P60T] was Constructed as Follows:

Strand overlapping extension PCR was performed using pJB02G-CSF[C17A] as the template. Primer JCB128 and JCB136 served as the A-B primers and JCB137 and JCB129 served as the C-D primers. The full-length mutated cDNA was prepared as described previously using the JCB128 and JCB129 primers. The B and C primers contained mismatched sequences such that a HpaI site was created and the protein expressed from the full-length sequence contained a consensus sequence for N-linked glycosylation in region 2 of the protein (see Table 1 and FIG. 1). The full-length cDNA was ligated back into the pCR2.1-Topo vector to create pCR2.1G-CSF[P57V,W58N,P60T] wherein the sequence was confirmed. G-CSF analog encoding DNA was then cloned into the Nhe/Xho sites of mammalian expression vector pJB02 (FIG. 3) to create pJB02G-CSF[P57V,W58N,P60T].

Preparation 1e: DNA Encoding G-CSF[P60N,S62T] was Constructed as Follows:

Strand overlapping extension PCR was performed using pJB02G-CSF[C17A] as the template. Primer JCB128 and JCB130 served as the A-B primers and JCB131 and JCB129 served as the C-D primers. The full-length mutated cDNA was prepared as described previously using the JCB128 and JCB129 primers. The B and C primers contained mismatched sequences such that a SpeI site was created and the protein expressed from the full-length sequence contained a consensus sequence for N-linked glycosylation in region 4 of the protein (see Table 1 and FIG. 1). The full-length cDNA was ligated back into the pCR2.1-Topo vector to create pCR2.1G-CSF[P60N,S62T] wherein the sequence was confirmed. G-CSF analog encoding DNA was then cloned into the Nhe/Xho sites of mammalian expression vector pJB02 (FIG. 3) to create pJB02G-CSF[P60N,S62T].

Preparation 1f: DNA Encoding G-CSF[S63N,P65T] was Constructed as Follows:

Strand overlapping extension PCR was performed using pJB02G-CSF[C17A] as the template. Primer JCB128 and JCB132 served as the A-B primers and JCB133 and JCB129 served as the C-D primers. The full-length mutated cDNA was prepared as described previously using the JCB128 and JCB129 primers. The B and C primers contained mismatched sequences such that a MfeI site was created and the protein expressed from the full-length sequence contained a consensus sequence for N-linked glycosylation in region 7 of the protein (see Table 1 and FIG. 1). The full-length cDNA was ligated back into the pCR2.1-Topo vector to create pCR2.1G-CSF[S63N,P65T] wherein the sequence was confirmed. G-CSF analog encoding DNA was then cloned into the Nhe/Xho sites of mammalian expression vector pJB02 (FIG. 3) to create pJB02G-CSF[S63N,P65T].

Preparation 1g: DNA Encoding G-CSF[Q67N,L69T] was Constructed as Follows:

Strand overlapping extension PCR was performed using pJB02G-CSF[C17A] as the template. Primer JCB134 and JCB138 served as the A-B primers and JCB139 and JCB135 served as the C-D primers. The full-length mutated cDNA was prepared as described previously using the JCB128 and JCB129 primers. The B and C primers contained mismatched sequences such that a NaeI site was created and the protein expressed from the full-length sequence contained a consensus sequence for N-linked glycosylation in region 9 of the protein (see Table 1 and FIG. 1). The full-length cDNA was ligated back into the pCR2.1-Topo vector to create pCR2.1G-CSF[Q67N,L69T] wherein the sequence was confirmed. G-CSF analog encoding DNA was then cloned into the Nhe/Xho sites of mammalian expression vector pJB02 (FIG. 3) to create pJB02G-CSF[Q67N,L69T].

Preparation 1h: DNA Encoding G-CSF[E93N,I95T] was Constructed as Follows:

Strand overlapping extension PCR was performed using pJB02G-CSF[C17A] as the template. Primer JCB134 and JCB140 served as the A-B primers and JCB141 and JCB135 served as the C-D primers. The full-length mutated cDNA was prepared as described previously using the JCB128 and JCB129 primers. The B and C primers contained mismatched sequences such that a BspEI site was created and the protein expressed from the full-length sequence contained a consensus sequence for N-linked glycosylation in region 10 of the protein (see Table 1 and FIG. 1). The full-length cDNA was ligated back into the pCR2.1-Topo vector to create pCR2.1G-CSF[E93N,I95T] wherein the sequence was confirmed. G-CSF analog encoding DNA was then cloned into the Nhe/Xho sites of mammalian expression vector pJB02 (FIG. 3) to create pJB02G-CSF[E93T,I95T].

Preparation 1i: DNA Encoding G-CSF[T133N,G135T] was Constructed as Follows:

Strand overlapping extension PCR was performed using pCR2.1G-CSF[C17A] as the template. Primer CF177 and T133Nrev served as the A-B primers and T133Nfor and CF176 served as the C-D primers. The full-length mutated cDNA was prepared as described previously using the CF177 and CF176 primers. The B and C primers contained mismatched sequences such that an Eco47III site was created and the protein expressed from the full-length sequence contained a consensus sequence for N-linked glycosylation in region 13 of the protein (see Table 1 and FIG. 1). The full-length cDNA was ligated back into the pCR2.1-Topo vector to create pCR2.1G-CSF[T133N,G135T] wherein the sequence was confirmed.

Preparation 1j: DNA Encoding G-CSF[A141N,A143T] was Constructed as Follows:

Strand overlapping extension PCR was performed using pCR2.1G-CSF[C17A] as the template. Primer CF177 and A141Nrev served as the A-B primers and A141Nfor and CF176 served as the C-D primers. The full-length mutated cDNA was prepared as described previously using the CF177 and CF176 primers. The B and C primers contained mismatched sequences such that an SapI site was created and the protein expressed from the full-length sequence contained a consensus sequence for N-linked glycosylation in region 14 of the protein (see Table 1 and FIG. 1). The full-length cDNA was ligated back into the pCR2.1-Topo vector to create pCR2.1G-CSF[A141N,A143T] wherein the sequence was confirmed.

Preparation 1k: DNA Encoding G-CSF[A37N,Y39T,T133N, G135T] was Constructed as Follows:

A 210 bp insert containing G-CSF[A37N,Y39T] was isolated from pCR2.1G-CSF[A37N,Y39T] using EcoNI. This fragment was ligated into pCR2.1G-CSF[T133N,G135T] which was prepared by cleavage with EcoNI and subsequent isolation of the vector (4359 bp) from a 210 bp fragment containing wild-type G-CSF sequences. This ligation created pCR2.1G-CSF[A37N,Y39T,T133N,G135T]. Analog encoding DNA was then subcloned into pJB02 (FIG. 3) using NheI/XhoI to create pJB02G-CSF[A37N,Y39T,T133N,G135T].

Preparation 1l: DNA Encoding G-CSF[A37N,Y39T,A141N, A143T] was Constructed as Follows:

A 210 bp insert containing G-CSF[A37N,Y39T] was isolated from pCR2.1G-CSF[A37N,Y39T] using EcoNI. This fragment was ligated into pCR2.1G-CSF[A141N,A143T] which was prepared by cleavage with EcoNI and subsequent isolation of the vector (4359 bp) from a 210 bp fragment containing wild-type G-CSF sequences. This ligation created pCR2.1G-CSF[A37N,Y39T,A141N,A143T]. Analog encoding DNA was then subcloned into pJB02 (FIG. 3) using NheI/XhoI to create pJB02G-CSF[A37N,Y39T,A141N,A143T].

Preparation 1m: DNA Encoding G-CSF[A37N,Y39T,P57V,W58N, P60T] was Constructed as Follows:

DNA encoding G-CSF[A37N,Y39T] was subcloned into pJB02 to create pJB02G-CSF[A37N,Y39T] and pJB02G-CSF[A37N,Y39T] served as the template for strand overlapping expression PCR. JCB128 and JCB136 served as the A and B primers and JCB137 and JCB129 served as the C and D primers. The full-length mutated cDNA was prepared as described previously using JCB128 and JCB129 primers. The resulting full-length DNA encodes a protein with consensus N-linked glycosylation sites in region 1 and region 2 of the protein (See Table 1 and FIG. 1). The full-length cDNA was ligated back into pCR2.1-Topo to create pCR2.1G-CSF[A37N,Y39T,P57V,W58N,P60T].

Preparation 1n: DNA Encoding G-CSF[A37N,Y39T,Q67N,L69T] was Constructed as Follows:

DNA encoding G-CSF[A37N,Y39T] was subcloned into pJB02 to create pJB02G-CSF[A37N,Y39T] and pJB02G-CSF[A37N,Y39T] served as the template for strand overlapping expression PCR. JCB134 and JCB138 served as the A and B primers and JCB139 and JCB135 served as the C and D primers. The full-length mutated cDNA was prepared as described previously using JCB128 and JCB129 primers. The resulting full-length DNA encodes a protein with consensus N-linked glycosylation sites in region 1 and region 9 of the protein (See Table 1 and FIG. 1). The full-length cDNA was ligated back into pCR2.1-Topo to create pCR2.1G-CSF[A37N,Y39T,Q67N,L69T].

Preparation 1o: DNA Encoding G-CSF[A37N,Y39T,E93N,I95T] was Constructed as Follows:

DNA encoding G-CSF[A37N,Y39T] was subcloned into pJB02 to create pJB02G-CSF[A37N,Y39T] and pJB02G-CSF[A37N,Y39T] served as the template for strand overlapping expression PCR. JCB134 and JCB140 served as the A and B primers and JCB141 and JCB135 served as the C and D primers. The full-length mutated cDNA was prepared as described previously using JCB128 and JCB129 primers. The resulting full-length DNA encodes a protein with consensus N-linked glycosylation sites in region 1 and region 10 of the protein (See Table 1 and FIG. 1). The full-length cDNA was ligated back into pCR2.1-Topo to create pCR2.1G-CSF[A37N,Y39T,E93N,I95T].

Preparation 1p: DNA Encoding G-CSF[A37N,Y39T, P57V,W58N,P60T,Q67N,L69T] was Constructed as Follows:

DNA encoding G-CSF[A37N,Y39T,Q67N,L69T] was subcloned into pJB02 to create pJB02G-CSF[A37N,Y39T,Q67N,L69T] and pJB02G-CSF[A37N,Y39T,P57V,W58N,P60T] served as the template for strand overlapping expression PCR. JCB155 and JCB136 served as the A and B primers and JCB137 and JCB135 served as the C and D primers. The full-length mutated cDNA was prepared as described previously using JCB155 and JCB134 primers. The resulting full-length DNA encodes a protein with consensus N-linked glycosylation sites in region 1, region 2, and region 9 of the protein (See Table 1 and FIG. 1). The full-length cDNA was ligated back into pCR2.1-Topo to create pCR2.1G-CSF[A37N,Y39T,P57V,W58N,P60T, Q67N,L69T].

Preparation 1q: DNA Encoding G-CSF[A37N,Y39T, S63N,P64T,E93N,I95T] was Constructed as Follows:

DNA encoding G-CSF[A37N,Y39T,E93N,I95T] was subcloned into pJB02 to create pJB02G-CSF[A37N,Y39T,E93N,I95T] and pJB02G-CSF[A37N,Y39T,E93N,I95T] served as the template for strand overlapping expression PCR. JCB155 and JCB132 served as the A and B primers and JCB133 and JCB135 served as the C and D primers. The full-length mutated cDNA was prepared as described previously using JCB155 and JCB135 primers. The resulting full-length DNA encodes a protein with consensus N-linked glycosylation sites in region 1, region 7, and region 10 of the protein (See Table 1 and FIG. 1). The full-length cDNA was ligated back into pCR2.1-Topo to create pCR2.1G-CSF[A37N,Y39T,S63N,P64T,E93N,I95T].

### EXAMPLE 2

### Expression of Glycosylated G-CSF Analogs

2a: Expression in 293/EBNA Cells:

Each full-length DNA encoding a G-CSF analog was subcloned into the NheI/XhoI sites of mammalian expression vector pJB02 (FIG. 3). This vector contains both the Ori P and Epstein Barr virus nuclear antigen (EBNA) components which are necessary for sustained, transient expression in 293 EBNA cells. This expression plasmid contains a puromycin resistance gene expressed from the CMV promoter as well as an ampicillin resistance gene. The gene of interest is also expressed from the CMV promoter.

The transfection mixture was prepared by mixing 73 μl of the liposome transfection agent Fugene 6® (Roche Molecular Biochemicals, Cat. No. 1815-075) with 820 μl Opti-Mem® (GibcoBRL Cat. No. 31985-062). G-CSF pJB02 DNA (12 μg), prepared using a Qiagen plasmid maxiprep kit (Qiagen, Cat. No. 12163), was then added to the mixture. The mixture was incubated at room temperature for 15 minutes.

Cells were plated on 10 cm² plates in DMEM/F12 3:1 (GibcoBRL Cat. No. 93-0152DK) supplemented with 5% fetal bovine serum, 20 mM HEPES, 2 mM L-glutamine, and 50 μg/mL Geneticin such that the plates were 60% to 80% confluent by the time of the transfection. Immediately before the transfection mixture was added to the plates, fresh media was added. The mixture was then added dropwise to cells with intermittent swirling. Plates were then incubated at 37° C. in a 5% CO₂ atmosphere for 24 hours at which point the media was changed to hybritech medium without serum. The media containing a secreted form of a glycosylated G-CSF analog was then isolated 48 hours later.

2b: Expression in CHO Cells:

The expression vector for expression in CHO-K1 cells pEE14.1 is illustrated in FIG. 4. This vector includes the glutamine synthetase gene which enables selection using methionine sulfoximine. This gene includes two poly A signals at the 3′ end. G-CSF analogs are expressed from the CMV promoter which includes 5′ untranslated sequences from the hCMV-MIE gene to enhance mRNA levels and translatability. The SV40 poly A signal is cloned 3′ of the G-CSF analog DNA. The SV40 late promoter drives expression of GS minigene. This expression vector encoding the gene of interest was prepared for transfection using a QIAGEN Maxi Prep Kit (QIAGEN, Cat. No. 12362). The final DNA pellet (50-100 μg) was resuspended in 100 μl of basal formulation medium (GibcoBRL CD-CHO Medium without L-Glutamine, without thymidine, without hypoxanthine).

Before each transfection, CHO-K1 cells were counted and checked for viability. A volume equal to 1×10⁷ cells was centrifuged and the cell pellet rinsed with basal formulation medium. The cells were centrifuged a second time and the final pellet resuspended in basal formulation medium (700 μl final volume).

The resuspended DNA and cells were then mixed together in a standard electroporation cuvette (Gene Pulsar Cuvette) used to support mammalian transfections, and placed on ice for five minutes. The cell/DNA mix was then electroporated in a BioRad Gene Pulsar device set at 300V/975 μF and the cuvette placed back on ice for five minutes. The cell/DNA mixed was then diluted into 20 ml of cell growth medium in a non-tissue culture treated T75 flask and incubated at 37° C./5% CO₂ for 48-72 hours.

The cells were counted, checked for viability, and plated at various cell densities in selective medium in 96 well tissue culture plates and incubated at 37° C. in a 5% CO₂ atmosphere. Selective medium is basal medium with 1×HT Supplement (GibcoBRL 100×HT Stock), 100 μg/mL Dextran Sulfate (Sigma 100 mg/ml stock), 1×GS Supplements (JRH BioSciences 50× Stock) and 25 μM MSX (Methionine Sulphoximine). The plates were monitored for colony formation and screened for glycosylated G-CSF analog production.

The expression vector for expression in CHO-DG44 cells PCID is illustrated in FIG. 5. The CMV promoter is used to drive expression of the G-CSF analog gene of interest. An IRES-DHFR sequence inserted downstream of that gene to allow for bicistronic expression. The DHFR gene confers methotrexate resistance on transfected cells. The BGH polyA signal is used to stabilize the mRNA resulting from the transcription of the cloned G-CSF analog gene. The DNA was prepared as described above.

Transfection conditions for CHO-DG44 cells (obtained from L. Chasin, Columbia University) were similar to those used for CHO-K1 cells except that the basal formulation medium consisted of JRH BioSciences ExCell 302 without L-Glutamine, without thymidine, and without hypoxanthine (Cat. No. 14312-79P) and the Cell/DNA mix was electroporated at 300V/975 μF or 400V/900 μF. Selection medium consisted of Basal Medium with the following additives: 6 mM L-Glutamine, 100 μg/mL Dextran Sulfate (Sigma 100 mg/ml stock) at various concentrations of Methotrexate (MTX).

### EXAMPLE 3

### Purification of Glycosylated G-CSF Analogs

An analytical reverses-phase system (Zorbax C8, 300SB, 0.46 cm×5 cm; solvent A; 0.1% TFA, Solvent B: 0.1% TFA/ACN, gradient: 10% A to 90% B in 15 min, 1 ml/min; detection at 280 nm) was used to estimate titers and examine hetero-geneity of the G-CSF analog being expressed. A standard curve was generated using known amounts of Neupogen®. The titer and yield (volume of conditioned medium) were used to establish column volumes, flow rates, etc.

The conditioned medium obtained, either from 293/EBNA cells (suspension or adherent) or CHO cells (K1 or DG44, stable or transient), was concentrated 2 to 10 fold and dialyzed versus 20 mM Tris, pH 7.5. An anion exchange column (either a prepacked HiTrap Q, or packed with Q fast flow Sepharose resin (both from Pharmacia) was equilibrated with 20 mM Tris, pH 7.5 and the dialyzed material loaded (1-2 CV/min depending on the volume of load and column). The protein was eluted from the column using a linear gradient from 0 to 400 mM NaCl over 40 min, at 1 CV/min and elution monitored by UV absorbance at 280 nm. SDS-PAGE analysis (and occasionally immunoblotting) or analytical reversed-phase on the eluant was used to identify fractions of interest, which were then pooled and dialyzed in 25 mM NaOAc, pH 4.0.

Appropriate HiTrap SP or Fast Flow Sepharose resin-packed columns (Pharmacia) were equilibrated with 25 mM NaOAc, pH 4.0, and the dialyzed anion exchange pool was applied. Similar flow rates and gradients as described for the anion exchange column chromatography were used.

The cation exchange step allowed for resolution of different isoforms which were analyzed further by IEF or oligosaccharide analysis. Some analogs were subjected to an additional purification step involving fractionation on a Mono Q anion exchange column or were subjected to size exclusion chromatography.

### EXAMPLE 4

### Characterization of Glycosylated G-CSF Analogs by MALDI-MS

All experiments were performed on a Micromass TofSpec-2E mass spectrometer fitted with Time Lag Focusing electronics, a Reflectron and Post Acceleration Detector (or P.A.D., used for high mass detection) The effective path length of the instrument in Linear mode is 1.2 meters, in Reflectron mode it is 2.3 meters.

Two dual micro-channel plate detectors are fitted for linear and reflectron mode detection. The laser used is a Laser Science Inc. VSL-337i nitrogen laser operating at 337 nm at 10 laser shots per second. All data were acquired using a 500 Mhz, 8 bit transient recorder and up to 100 laser shots were averaged per spectrum using the Post Acceleration Detector (when necessary to increase ion signal).

The detection efficiency of a micro-channel plate or electron multiplier reduces as the ion mass increases. The operation of these devices relies on the production of secondary electrons from the ion bombardment of a surface and this becomes less efficient as the ion impact velocity reduces. Higher mass ions have lower velocities than low mass ions with the same energy and hence produce less, or no, secondary ions. In order to enhance the detection of high mass ions previous studies have shown that the secondary ion species may be accelerated from a dynode surface place in the ion path into a conventional electron multiplier. The TofSpec-2E has been modified such that an ion-to-ion conversion dynode may be moved in and out of position in front of the standard micro-channel plate detector.

The net effect of the introduction of this ion-to-ion conversion dynode is a small increase in the single mass peak width and so the ability to move the dynode out of the ion path ensures that the resolution at low mass, where the detection efficiency is already high, need not be compromised.

Sinapinic acid was used as the ionization matrix as all masses observed were above 10 kDa. Mass appropriate reference proteins were used for internal and external calibration files in order to obtain accurate mass determinations for the samples analyzed. Samples were all analyzed using a 1:2 sample to matrix dilution.

The instrument was initially set up under the following linear high mass detector conditions:

| | | | |
|---|---|---|---|
| Source Voltage: | 20.0 keV | Pulse Voltage: | 3.0 keV |
| Extraction Voltage: | 20.0 keV | Laser Coarse: | 50 |
| Focus Voltage: | 16.0 keV | Laser Fine: | 50 |
| Linear detector: | 3.7 keV | | |
| P.A.D.: | (off line) | | |

These settings were then modified (if needed) to give the best signal/noise ratio and highest resolution. Table 2 provides a characterization of different glycosylated G-CSF analogs.

TABLE 2

| | Non-glycosylated | |
|---|---|---|
| Protein | Mass | Determined Mass |
| Neupogen ® | 18798.9 | 18800.7 |
| Wild-type | 18667.67 | 18966 |
| | | 19258. 6 |
| | | 19526.6 |
| G-CSF[C17A] | 18635.61 | 18958.9 |
| | | 19248.3 |
| | | 19523.8 |
| G-CSF[T133N, G135T] | 18692.66 | 18690.3 |
| | | 20.9 (20.3-21.5) |
| G-CSF[A141N, A143T] | 18708.66 | 19699.6 (18.5-21) |
| | | 21.7 (21-22.5) |
| G-CSF[A37N, Y39T] | 18616.56 | 19.6 (19-21) |
| | | 21.7 (21-22.5) |
| G-CSF[P57V, W58N, P60T] | 18571.5 | 21502.4 (20.5-22.6) |
| G-CSF[Q67N, L69T] | 18611.5 | 21569.1 (20.2-22.9) |
| G-CSF[E93N, I95T] | 18610.6 | 21780.7 (20.5-23) |
| G-CSF[A37N, Y39T, T133N, | 18673.62 | 18678.5 |
| G135T] | | 20.8, 23 (20-26) |
| G-CSF[A37N, Y39T, A141N, | 18689.62 | 19633.7 |
| A143T] | | 21.8, 23.7 (20.5-26.5) |
| G-CSF[P60N, S62T] | 18666.62 | 19612.9 |
| | | 21.5, 21.6, |
| | | 21.7 (20.5-24) |
| G-CSF[S63N, P65T] | 18666.62 | 19619.3 |
| | | 21.5, 21.6, 21.8 |
| | | (20.5-24) |
| G-CSF[A37N, Y39T, P57V, | 18550.46 | 19.6, 23589.5 |
| W58N, P60T] | | (22-25) |
| G-CSF[A37N, Y39T, Q67N, | 18590.48 | 19.6, 21.5 (20.5-22), |
| L69T] | | 23614.4 (22-25) |
| G-CSF[A37N, Y39T, E93N, | 18589.50 | 24859.2 (22.5-27.5) |
| I95T] | | |
| G-CSF[A37N, Y39T, P57V, | 18524.38 | 19.5, |
| W58N, P6OT, Q67N, L69T] | | 26073.6 (22.5-30) |
| G-CSF[A37N, Y39T, S63N, | 18620.51 | 19.5, 21.5 (20-22.5), |
| P65T, E93N, I95T] | | 26642.4 |
| | | (22.5-30) |

The mass range encompassed by the broad peak is given in brackets and the centroid of the peak is indicated outside the bracket. The mass of the prominent peak is shown in bold. Note that the centroid peak and bracketed mass ranges are provided in Kilo-Daltons (KDa) whereas the masses in column 2 and the prominent peak masses are provided in Daltons (Da). The difference in the mass between columns 2 and 3 is indicative of the mass of added carbohydrate. Proof that the measured peaks represented G-CSF was obtained from immunoblots (Example 5).

### EXAMPLE 5

### Characterization of Glycosylated G-CSF Analogs Using SDS-PAGE

SDS-PAGE followed by immunoblotting was used to analyze the conditioned medium from cells transfected with various G-CSF analog expression vectors. SDS-PAGE was performed on a Novex Powerease 500 system using Novex 16% Tris-Glycine Precast gels (EC6498), running buffer (10×, LC2675) and sample buffer (L2676). Samples were reduced with 50 mM DTT and heated 3-5 min at 95° C. prior to loading.

After running the SDS-PAGE gel, water and transfer buffer (1× Tris-Glycine Seprabuff (Owl Scientific Cat. No. ER26-S) with 20% methanol) were used to rinse SDS from the gels. A Novex transfer apparatus was used with PVDF (BioRad, Cat. No. 162-0174) and nitrocellulose membranes (BioRad, Cat. No. 1703965 or 1703932). Transfer was carried out at room temperature for 90 min at 30-35 V. Membranes were blocked in X PBS with 0.1% Tween-20 (Sigma, Cat. No. P-7949) and 5% Milk (BioRad, Cat. No. 170-6404) for 1-12 hours at 4° C. Antibodies are diluted into 1×PBS+5% Milk and the blots are incubated in these solutions for 1-2 h at 4° C. Between incubations, the blots are washed four times for 5 min each with 1×PBS+0.2% Tween-20 at room temperature. PBS was made from either GIBCO 10×PBS (Cat No. 70011), to give a final composition of 1 mM monobasic potassium phosphate, 3 mM dibasic sodium phosphate, 153 mM sodium chloride, pH 7.4, or PBS pouches from Sigma (Cat. No. 1000-3), to give 120 mM NaCl, 2.7 mM KCl and 10 mM phosphate, pH 7.4 at 25° C.

The primary antibody was a polycolonal anti-human G-CSF purchased from R&D Systems (Cat No. AF-214-NA). The antibody was diluted in 1×PBS to a concentration of 0.1 mg/mL and stored in aliquots at −20° C. Aliquots were diluted 1:600 before use. The secondary antibody was an anti-goat IgG (H+L) peroxidase conjugate affinity purified from swine (Roche, Cat No. 605275). The secondary antibody was diluted 1:5000. An ECL system (Amersham Pharmacia Biotech, Cat. No. RN2108 and Cat. No. RPN1674H) was used for developing blots.

Conditioned medium (25 to 100 ul) was used for each lane. FIG. 6a shows the increase in mobility accompanying the introduction of additional sites for glycosylation. Lanes 4 and 5 represent G-CSF analogs that have a single consensus glycosylation site added. Lanes 6 and 7 represent G-CSF analogs that have two consensus glycosylation sites added. FIG. 6b shows the migration pattern of CHO-K1 conditioned media from G-CSF analog transfected cells. Lanes 1 through 4 represent different triple glycosylated G-CSF analogs. The blot confirms addition of glycosylation as seen by the diffussness of the bands and also the increased mass.

Several orthogonal methods are available to verify addition of N-linked sugars to the protein. Samples are treated with N-glycanase and run alongside nontreated samples on SDS-PAGE gels. Treated samples migrate faster than nontreated ones due to release of N-linked sugars. Alternatively, purified protein is subjected to oligoprofiling or LC/MS.

Oligoprofiling as described in Kanazawa et al (1999, Biol. Pharm. Bull., 22, 339-346) was used to compare extent of sialylation and branching. Enzymatically released N-linked oligosaccharides were labeled with 2-aminobenzamide and analyzed by weak anion exchange on DEAE-5PW column (7.5×0.75 cm, Tosohaas). Detailed characterization of sugars was performed by subjecting purified protein to reduction, alkylation and endoproteinase digestion (e.g. with thermolysin or Glu-C digestion). LC/MS analysis is carried out on the digests prior to and after neuraminidase treatment.

Numerous GCSF analogs were purified as described in Example 3 and subjected to protease and neuraminidase treatment and LC/MS analysis wherein the presence of N-linked sugars was confirmed.

### EXAMPLE 6

### Activity of Glycosylated G-CSF Analogs

Activity assays were performed using cells expressing the G-CSF receptor and stably transfected with a reporter plasmid. The reporter is expressed in response to cellular signals generated when the receptor is occupied by ligand in the appropriate conformation.

The cells described above were maintained in growth media (DMEM containing 10% FBS, 25 mM Hepes, 50 ug/ml gentamicin, 500 ug/ml G418 and 100 ug/ml hygromicin B). Cells were trypsinized, and seeded in Biocoat 96 well poly-D-lysine (PDL) white/clear plates (Becton-Dickinson) at 40,000 cells/well and serum-starved overnight in DMEM with 0.5% FBS, 25 mM Hepes, and 50 ug/ml gentamicin. The next morning the media was replaced and compounds added for a 5 hour incubation at 37° C. Media was aspirated, cells were washed once with PBS and then lysed. Data were generated in triplicate, and mean reporter activity was expressed as fold induction over the unstimulated control. The assay was qualitatively and quantitatively validated by testing the response as a function of cell culture passages and exposure to commercially available G-CSF. EC50 values relative to Neupogen® are illustrated in Table 3.

TABLE 3
Activity of glycosylated G-CSF analogs

| Protein | EC50/Neupogen |
|---|---|
| Wild-type G-CSF | 0.38 |
| G-CSF[C17A] | 0.66 |
| G-CSF[T133N, G135T] | 0.37 |
| G-CSF[A141N, A143T] | 0.54 |
| G-CSF[A37N, Y39T] | 0.80 |
| G-CSF[P60N, S62T] | 0.94 |
| G-CSF[S63N, P65T] | 0.9 |
| G-CSF[P57V, W58N, P60T] | 0.59 |
| G-CSF[Q67N, L69T] | 0.78 |
| G-CSF[E93N, I95T] | 0.36 |
| G-CSF[A37N, Y39T, P57V, W58N, | 0.53 |
| Q67N, L69T] | |
| G-CSF[A37N, Y39T, S63N, P65T, | 0.97 |
| E93N, I95T] | |

### EXAMPLE 7

### In Vivo Assay

Purified and characterized G-CSF analogs are injected into mice and/or monkeys as a single subcutaneous injection. Groups of BDF mice (normal or splenectomized) or monkeys are injected with vehicle or GCSF analog subcutaneously at doses ranging from 200 to 1000 ug/kg. In each group 4 to 6 animals are bled at 8 hour intervals. Serum levels of the analogs are followed by taking plasma at different timepoints and subjecting it to ELISA or in vitro bioactivity to measure protein levels. The samples are subjected to blood cell analyses to determine various blood cell parameters. Analogs will have extended half-lives as reflected by pharmacokinetic analysis and will show sustained duration of action leading to increased numbers of neutrophils for several days following the injection. This is in contrast to Neupogen which has a plasma half-life of 4 hours and neutrophil levels that increase 4 to 6 hours after injection, but return to baseline levels within 24 hours.

### EXAMPLE 8

### Stability Analysis Following Thermal or Denaturant Induced Unfolding

The thermal unfolding transition of glycosylated G-CSF analogs compated to wild-type G-CSF expressed in CHO cells was monitored by differential scanning calorimetry (DSC). Data was collected on a VP-DSC MicroCalorimeter using VPViewer software and Origin DSC software for data analysis. The matched sample and reference cells had a working volume of 0.5 mL. The protein samples were dialyzed against 25 mM sodium acetate, 100 mM NaCl, pH 4.5 buffer overnight and the concentration of protein was determined by analytical reverse-phase HPLC. Buffer was also run overnight in both cells to establish a thermal history prior to sample runs. Proteins were then diluted to approximately 0.4 mg/mL, and the dialysate was used as the reference solution. After degassing, both sample and reference were loaded in cells with 2.5 mL needle through a filling funnel. Pressure was kept at approximately 30 psi. with a pressure cap. Data was collected between 50 and 90° C., changing 1° C./min, after a 15 min equilibrium at 5° C.

TABLE 4
Midpoint of the transition temperature (Tm) of
glycosylated analogs

| | Tm ° C. |
|---|---|
| | (Standard |
| Protein (cell line) | Deviation) |
| G-CSF [WT], CHO | Precipitated |
| G-CSF [C17A], 293 EBNA | 60.6(1.4) |
| G-CSF [A37N, Y39T, Q67N, L69T], 293 | 61.7(1.7) |
| EBNA | |
| G-CSF [A37N, Y39T, E93N, I95T], 293 | 62(1.1) |
| EBNA | |
| G-CSF [A37N, Y39T, Q67N, L69T], CHO- | 64.8(0.65) |
| DG44 | |
| G-CSF [A37N, Y39T, S63N, P65T, E93N, | 69(2.4) |
| I95T], CHO K1 | |

Guanidine hydrochloride induced denaturation of glycosylated G-CSF analogs was also used as a measure of protein stability and determined based on ellipticity at 224 nm. as a function of denaturant concentration. JMP software was used for calculating the mid point of unfolding transition (M) and ΔG. Data was collected on an AVIV model 62DS spectrometer using a 0.5 cm pathlength cell at 25° C., with a 2 nm bandwidth. The mid point of unfolding transition for G-CSF [C17A](293 EBNA) was 3M, versus 3.4M for G-CSF [A37N, Y39T, S63N, P65T, E93N, 195T](CHO K1). The corresponding ΔG was 9 kcals/mole vs 10.5 kcals/mole, indicating increased stability of the latter.

We claim:

1. A G-CSF analog comprising a glycosylated protein wherein the glycosylated protein comprises an amino acid sequence of formula I: [SEQ ID NO:1]

| | | | | | | | | | | | | | | | | |
|---|---|---|---|---|---|---|---|---|---|---|---|---|---|---|---|---|
| 1 | | | | 5 | | | | | 10 | | | | | 15 | | |
| Thr | Pro | Leu | Gly | Pro | Ala | Ser | Ser | Leu | Pro | Gln | Ser | Phe | Leu | Leu | Lys | (I) |
| | | | 20 | | | | | 25 | | | | | 30 | | | |
| Xaa | Leu | Glu | Gln | Val | Arg | Lys | Ile | Gln | Gly | Asp | Gly | Ala | Ala | Leu | Gln | |
| | | 35 | | | | | 40 | | | | | 45 | | | | |
| Glu | Lys | Leu | Cys | Xaa | Xaa | Xaa | Lys | Leu | Cys | His | Pro | Glu | Glu | Leu | Val | |
| | 50 | | | | | 55 | | | | | 60 | | | | | |
| Leu | Leu | Gly | His | Ser | Leu | Gly | Ile | Xaa | Xaa | Xaa | Xaa | Xaa | Xaa | Xaa | Xaa | |
| 65 | | | | | 70 | | | | | 75 | | | | | 80 | |
| Xaa | Xaa | Xaa | Xaa | Xaa | Gln | Leu | Ala | Gly | Cys | Leu | Ser | Gln | Leu | His | Ser | |
| | | | | 85 | | | | | 90 | | | | | 95 | | |
| Gly | Leu | Phe | Leu | Tyr | Gln | Gly | Leu | Leu | Gln | Ala | Leu | Xaa | Xaa | Xaa | Ser | |
| | | | 100 | | | | | 105 | | | | | 110 | | | |
| Xaa | Glu | Leu | Gly | Pro | Thr | Leu | Asp | Thr | Leu | Gln | Leu | Asp | Val | Ala | Asp | |
| | | 115 | | | | | 120 | | | | | 125 | | | | |
| Phe | Ala | Thr | Thr | Ile | Trp | Glu | Gln | Met | Gln | Glu | Leu | Gly | Met | Ala | Pro | |
| | 130 | | | | | 135 | | | | | 140 | | | | | |
| Ala | Leu | Gln | Pro | Xaa | Xaa | Xaa | Ala | Met | Pro | Ala | Phe | Xaa | Xaa | Xaa | Phe | |
| 145 | | | | | 150 | | | | | 155 | | | | | 160 | |
| Gln | Arg | Arg | Ala | Gly | Gly | Val | Leu | Val | Ala | Ser | His | Leu | Aln | Ser | Phe | |
| | | | | 165 | | | | | 170 | | | | | | | |
| Leu | Glu | Val | Ser | Tyr | Arq | Val | Leu | Arg | His | Leu | Ala | Gln | Pro | | | |

wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gln or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;
and wherein:
Xaa at positions 37, 38, and 39 constitute region 1;
Xaa at positions 58, 59, and 60 constitute region 2;
Xaa at positions 59, 60, and 61 constitute region 3;
Xaa at positions 60, 61, and 62 constitute region 4;
Xaa at positions 61, 62, and 63 constitute region 5;
Xaa at positions 62, 63, and 64 constitute region 6;
Xaa at positions 63, 64, and 65 constitute region 7;
Xaa at positions 64, 65, and 66 constitute region 8;
Xaa at positions 67, 68, and 69 constitute region 9;
Xaa at positions 93, 94, and 95 constitute region 10;
Xaa at positions 94, 95, and Ser at position 96 constitute region 11;
Xaa at positions 95, and 97, and Ser at position 96 constitute region 12;
Xaa at positions 133, 134, and 135 constitute region 13;
Xaa at positions 141, 142, and 143 constitute region 14;
and provided that at least one of regions 1 through 14 comprises the sequence Asn Xaa1 Xaa2 wherein Xaa1 is any amino acid except Pro and Xaa2 is Ser or Thr.

2. The glycosylated protein of claim 1 wherein any two regions of regions 1 through 14 comprise the sequence Asn Xaa1 Xaa2 wherein Xaa1 is any amino acid except Pro and Xaa2 is Ser or Thr.

3. The glycosylated protein of claim 1 wherein any three regions of regions 1 through 14 comprise the sequence Asn Xaa1 Xaa2 wherein Xaa1 is any amino acid except Pro and Xaa2 is Ser or Thr.

4. The glycosylated protein of claim 1 wherein any four regions of regions 1 through 14 comprise the sequence Asn Xaa1 Xaa2 wherein Xaa1 is any amino acid except Pro and Xaa2 is Ser or Thr

5. The glycosylated protein of claim 1 wherein the protein is selected from the group consisting of:
a) G-CSF[A37N,Y39T]
b) G-CSF[P57V,W58N,P60T]
c) G-CSF[P60N,S62T]
d) G-CSF[S63N,P65T]
e) G-CSF[Q67N,L69T]
f) G-CSF[E93N,I95T]
g) G-CSF[T133N,G135T]
h) G-CSF[A141N,A143T]
i) G-CSF[A37N,Y39T,P57V,W58N,P60T]
j) G-CSF[A37N,Y39T,P60N,S62T]
k) G-CSF[A37N,Y39T,S63N,P65T]
l) G-CSF[A37N,Y39T,Q67N,L69T]
m) G-CSF[A37N,Y39T,E93N,I95T]
n) G-CSF[A37N,Y39T,T133N,G135T]
o) G-CSF[A37N,Y39T,A141N,A143T]
p) G-CSF[A37N,Y39T,P57V,W58N,P60T,S63N,P65T]
q) G-CSF[A37N,Y39T,P57V,W58N,P60T,Q67N,L69T]
r) G-CSF[A37N,Y39T,S63N,P65T,E93N,I95T]

6. A glycosylated protein which is the product of the expression in a host cell of an exogenous DNA sequence which comprises a DNA sequence encoding a protein of any one of claims 1 through 5.

7. An isolated nucleic acid sequence, comprising a polynucleotide encoding a protein of any one of claims 1 through 5.

8. An isolated nucleic acid sequence, comprising a polynucleotide which comprises a DNA sequence selected from the group consisting of:
a) SEQ ID NO:2
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC AAC CAG ACC GCC ATG CCG GCC TTC GCC TCT GCT TTC
CGG GAC GTC GGG TTG GTC TGG CGG TAC GGC CGG AAG CGG AGA CGA AAG
CZG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
b) SEQ ID NO:3
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
c) SEQ ID NO:4
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
d) SEQ ID NO:5
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
e) SEQ ID NO:6
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
f) SEQ ID NO:7
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
g) SEQ ID NO:8
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
h) SEQ ID NO:9
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
i) SEQ ID NO:10
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
j) SEQ ID NO:11
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
k) SEQ ID NO:12
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
l) SEQ ID NO:13
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
m) SEQ ID NO:14
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
n) SEQ ID NO:15
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
o) SEQ ID NO:16
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG

9. A vector comprising a nucleic acid sequence according to claim 7 or 8.

10. A host cell comprising the vector of claim 9.

11. A host cell expressing at least one protein of any one of claims 1 through 5.

12. The host cell of claim 10 or 11 wherein said host cell is a CHO cell.

13. A process for producing a glycosylated protein comprising the steps of transcribing and translating a polynucleotide of claims 7 or 8 under conditions wherein the protein is glycosylated and expressed in detectable amounts.

14. A method for increasing neutrophil levels in a mammal comprising the administration of a therapeutically effective amount of the glycosylated protein of any one of claims 1 through 5.

15. The use of the glycosylated protein as claimed in any one of claims 1 through 5 for the manufacture of a medicament for the treatment of patients with insufficient circulating neutrophil levels.

16. Use of a glycosylated protein of any one of claims 1 through 5 as a medicament.

17. Use of a glycosylated protein of any one of claims 1 through 5 for the treatment of patients with insufficient circulating neutrophil levels.

18. A pharmaceutical formulation adapted for the treatment of patients with insufficient neutrophil levels comprising a glycosylated protein of any one of claims 1 through 5.

19. A glycosylated protein as hereinbefore described with reference to any one of the Examples.


SEQUENCE LISTING (65 sequences)

SEQ ID NO: 1 - protein, 174 aa, Artificial Sequence
  synthetic construct
TPLGPASSLPQSFLLKXLEQVRKIQGDGAALQEKLCXXXKLCHPEELVLLGHSLGIXXXXXXXXXXXXXQLAGCLSQLHSGLFLYQGLLQALXXXSXELGPTLDTLQLDVADFATTIWQQMEELGMAPALQPXXXAMPAFXXXFQRRAGGVLVASHLQSFLEVSYRVLRHLAQP

SEQ ID NO: 2 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccaaccagaccgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 3 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcaactctaccttccagcgccgggcaggaggggtcctggttgcctcccatctgcagagcttcctggaggtgtcgtaccgcgtcttaaggcaccttgcccagccc

SEQ ID NO: 4 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 5 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtgccacctacaagctgtgccaccccgaggagctggtgctgctcggacactctctgggcatcccctgggctaacactagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 6 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtgccacctacaagctgtgccaccccgaggagctggtgctgctcggacactctctgggcatcccctgggctcccctgagcaattgcaccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 7 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtgccacctacaagctgtgccaccccgaggagctggtgctgctcggacactctctgggcatcgttaacgctaccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 8 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagcaacgccacccagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 9 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctgaacgggacctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 10 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccaaccagaccgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 11 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcaactctaccttccagcgccgggcaggaggggtcctggttgcctcccatctgcagagcttcctggaggtgtcgtaccgcgtcttaaggcaccttgcccagccc

SEQ ID NO: 12 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcgttaacgctaccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 13 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcccctgggctcccctgagcagctgccccagcaacgccacccagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 14 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctccaacggtaccggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 15 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcgttaacgctaccctgagcagctgccccagcaacgccacccagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 16 - DNA, 522 bp, Artificial Sequence
  synthetic construct
acccccctgggccctgccagctccctgccccagagcttcctgctcaaggccttagagcaagtgaggaagatccagggcgatggcgcagcgctccaggagaagctgtgtaacaccaccaagctgtgccaccccgaggagctggtgctgctcggacactctctgggcatcccctgggctcccctgagcaattgcaccagccaggccctgcagctggcaggctgcttgagccaactccatagcggccttttcctctaccaggggctcctgcaggccctgaacgggacctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgtcttaaggcaccttgcccagccc

SEQ ID NO: 17 - protein, 174 aa, Artificial Sequence
  synthetic construct
TPLGPASSLPQSFLLKALEQVRKIQGDGAALQEKLCNTTKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 18 - protein, 174 aa, Artificial Sequence
  synthetic construct
TPLGPASSLPQSFLLKALEQVRKIQGDGAALQEKLCATYKLCHPEELVLLGHSLGIVNATLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 19 - protein, 174 aa, Artificial Sequence
  synthetic construct
TPLGPASSLPQSFLLKALEQVRKIQGDGAALQEKLCATYKLCHPEELVLLGHSLGIPWANLTSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 20 - protein, 174 aa, Artificial Sequence
  synthetic construct
TPLGPASSLPQSFLLKALEQVRKIQGDGAALQEKLCATYKLCHPEELVLLGHSLGIPWAPLSNCTSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 21 - protein, 174 aa, Artificial Sequence
  synthetic construct
TPLGPASSLPQSFLLKALEQVRKIQGDGAALQEKLCATYKLCHPEELVLLGHSLGIPWAPLSSCPSNATQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 22 - protein, 174 aa, Artificial Sequence
  synthetic construct
TPLGPASSLPQSFLLKALEQVRKIQGDGAALQEKLCATYKLCHPEELVLLGHSLGIPWAPLSSCPSQALQLAGCLSQLHSGLFLYQGLLQALNGTSPELGPTLDTLQLDVADFATTIWQQMEELGMAPALQPTQGAMPAFASAFQRRAGGVLVASHLQSFLEVSYRVLRHLAQP

SEQ ID NO: 23 - protein, 174 aa, Artificial Sequence
  synthetic construct
TPLGPASSLPQSFLLKALEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPNQTAMPAFASAFQRRAGGVLVASHLQSFLEVSYRVLRHLAQP

SEQ ID NO: 24 - protein, 174 aa, Artificial Sequence
  synthetic construct
TPLGPASSLPQSFLLKALEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFNSTFQRRAGGVLVASHLQSFLEVSYRVLRHLAQP

SEQ ID NO: 25 - DNA, 69 bp, Artificial Sequence
  synthetic construct
gtaagcttgcgtcgacgctagcggcgcgccgccatggccggacctgccacccagagccccatgaagctg

SEQ ID NO: 26 - DNA, 61 bp, Artificial Sequence
  synthetic construct
ggggcagggagctggctgggcccagtggagtggcttcctgcactgtccagagtgcactgtg

SEQ ID NO: 27 - DNA, 59 bp, Artificial Sequence
  synthetic construct
ggacagtgcaggaagccactccactgggcccagccagctccctgccccagagcttcctg

SEQ ID NO: 28 - DNA, 72 bp, Artificial Sequence
  synthetic construct
gaacctcgaggatcctcattagggctgggcaaggtgccttaagacgcggtacgacacctccaggaagctctg

SEQ ID NO: 29 - DNA, 69 bp, Artificial Sequence; identical to SEQ ID NO: 25
  synthetic construct

SEQ ID NO: 30 - DNA, 57 bp, Artificial Sequence
  synthetic construct
gctctaaggccttgagcaggaagctctggggcagggagctcgctgggcccagtggag

SEQ ID NO: 31 - DNA, 53 bp, Artificial Sequence
  synthetic construct
gggcccagcgagctccctgccccagagcttcctgctcaaggccttagagcaag

SEQ ID NO: 32 - DNA, 72 bp, Artificial Sequence; identical to SEQ ID NO: 28
  synthetic construct

SEQ ID NO: 33 - DNA, 69 bp, Artificial Sequence; identical to SEQ ID NO: 25
  synthetic construct

SEQ ID NO: 34 - DNA, 61 bp, Artificial Sequence
  synthetic construct
gtccgagcagcactagttcctcggggtggcacagcttggtggtgttacacagcttctcctg

SEQ ID NO: 35 - DNA, 66 bp, Artificial Sequence
  synthetic construct
ggcgcagcgctccaggagaagctgtgtaacaccaccaagctgtgccaccccgaggaactagtgctg

SEQ ID NO: 36 - DNA, 72 bp, Artificial Sequence; identical to SEQ ID NO: 28
  synthetic construct

SEQ ID NO: 37 - DNA, 69 bp, Artificial Sequence; identical to SEQ ID NO: 25
  synthetic construct

SEQ ID NO: 38 - DNA, 61 bp, Artificial Sequence
  synthetic construct
gcccggcgctggaaagcgctggcgaaggccggcatggcggtctggttgggctgcagggcag

SEQ ID NO: 39 - DNA, 60 bp, Artificial Sequence
  synthetic construct
ggcccctgccctgcagcccaaccagaccgccatgccggccttcgccagcgctttccagcg

SEQ ID NO: 40 - DNA, 72 bp, Artificial Sequence; identical to SEQ ID NO: 28
  synthetic construct

SEQ ID NO: 41 - DNA, 69 bp, Artificial Sequence; identical to SEQ ID NO: 25
  synthetic construct

SEQ ID NO: 42 - DNA, 68 bp, Artificial Sequence
  synthetic construct
gcccggcgctggaaggtagagttgaaggccggcatggcaccctgggtgggctgaagagcaggggccat

SEQ ID NO: 43 - DNA, 74 bp, Artificial Sequence
  synthetic construct
gggaatggcccctgctcttcagcccacccagggtgccatgccggccttcaactctaccttccagcgccgggcag

SEQ ID NO: 44 - DNA, 72 bp, Artificial Sequence; identical to SEQ ID NO: 28
  synthetic construct

SEQ ID NO: 45 - DNA, 20 bp, Artificial Sequence
  synthetic construct
gctagcggcgcgccaccatg

SEQ ID NO: 46 - DNA, 33 bp, Artificial Sequence
  synthetic construct
gctcagggtagcgttaacgatgcccagagagtg

SEQ ID NO: 47 - DNA, 30 bp, Artificial Sequence
  synthetic construct
gggcatcgttaacgctaccctgagcagctg

SEQ ID NO: 48 - DNA, 27 bp, Artificial Sequence
  synthetic construct
gactcgaggatcctcattagggctggg

SEQ ID NO: 49 - DNA, 38 bp, Artificial Sequence
  synthetic construct
gctagcggcgcgccaccatggccggacctgccacccag

SEQ ID NO: 50 - DNA, 45 bp, Artificial Sequence
  synthetic construct
caagcagccggccagctgggtggcgttgctggggcagctgctcag

SEQ ID NO: 51 - DNA, 37 bp, Artificial Sequence
  synthetic construct
gccccagcaacgccacccagctggccggctgcttgag

SEQ ID NO: 52 - DNA, 47 bp, Artificial Sequence
  synthetic construct
gactcgaggatcctcattagggctgggcaaggtgccttaagacgcgg

SEQ ID NO: 53 - DNA, 20 bp, Artificial Sequence
  synthetic construct
gctagcggcgcgccaccatg

SEQ ID NO: 54 - DNA, 27 bp, Artificial Sequence
  synthetic construct
ggggcaactagtcaggttagcccaggg

SEQ ID NO: 55 - DNA, 27 bp, Artificial Sequence
  synthetic construct
gctaacctgactagttgccccagccag

SEQ ID NO: 56 - DNA, 27 bp, Artificial Sequence
  synthetic construct
gactcgaggatcctcattagggctggg

SEQ ID NO: 57 - DNA, 20 bp, Artificial Sequence
  synthetic construct
gctagcggcgcgccaccatg

SEQ ID NO: 58 - DNA, 25 bp, Artificial Sequence
  synthetic construct
ggtgcaattgctcaggggagcccag

SEQ ID NO: 59 - DNA, 23 bp, Artificial Sequence
  synthetic construct
gcaattgcaccagccaggccctg

SEQ ID NO: 60 - DNA, 27 bp, Artificial Sequence
  synthetic construct
gactcgaggatcctcattagggctggg

SEQ ID NO: 61 - DNA, 38 bp, Artificial Sequence; identical to SEQ ID NO: 49
  synthetic construct

SEQ ID NO: 62 - DNA, 37 bp, Artificial Sequence
  synthetic construct
ccggactggtcccgttcagggcctgcaggagcccctg

SEQ ID NO: 63 - DNA, 35 bp, Artificial Sequence
  synthetic construct
gaacgggaccagtccggagttgggtcccaccttgg

SEQ ID NO: 64 - DNA, 47 bp, Artificial Sequence; identical to SEQ ID NO: 52
  synthetic construct

SEQ ID NO: 65 - DNA, 36 bp, Artificial Sequence
  synthetic construct
gtcgacgctagcggcgcgccaccatggccggacctg